(19) United States
(12) Patent Application Publication
(10) Pub. No.: US 2024/0342220 A1
(43) Pub. Date: Oct. 17, 2024

(54) COMPOSITIONS AND METHODS FOR CELL-BASED DELIVERY SYSTEMS

(71) Applicants: Washington University, St. Louis, MO (US); Shriners Hospitals for Children, Tampa, FL (US)

(72) Inventors: Farshid Guilak, St. Louis, MO (US); Lara Pferdehirt, St. Louis, MO (US)

(21) Appl. No.: 18/284,487

(22) PCT Filed: Mar. 31, 2022
(86) PCT No.: PCT/US22/22904

§ 371 Date: Sep. 27, 2023

Related U.S. Application Data
(60) Provisional application No. 63/168,857, filed on Mar. 31, 2021.

(51) Int. Cl. A61K 35/32; A61K 38/20; C07K 14/47; C07K 14/545; C12N 15/86

(52) U.S. Cl. CPC A61K 35/32; A61K 38/2006; C07K 14/4702; C07K 14/545; C12N 15/86; C07K 2319/92; C12N 2740/15043

(57) ABSTRACT

Among the various aspects of the present disclosure is the provision of compositions and methods of making genetically modified cells comprising a synthetic circuit that is responsive to a circadian input to the cell and methods of use thereof. This disclosure uses a cells circadian rythym to provide gene-based delivery of biologic drugs at prescribed times, phases and frequencies. Once reprogrammed, the cells can be reimplanted in the body for this purpose.

### CROSS REFERENCE TO RELATED APPLICATION

This application claims priority to U.S. Provisional Application No. 63/168,857, filed Mar. 31, 2021, the disclosure of which is incorporated herein by reference.

### FIELD OF THE TECHNOLOGY

This disclosure generally relates to compositions and methods for cell therapy using engineered cells with timed delivery of a therapeutic. In particular, the disclosure provides cells engineered to use the cell's internal circadian rhythm to drive a gene output and methods of using the same for the treatment of a condition, disease or disorder (i.e. chronogenetic therapy). The synthetic circuit may involve any combination of nucleic acid molecules (e.g. gene regulatory regions) involved in regulating or responding to circadian rhythm signaling and used to drive expression of a therapeutic biologic.

### BACKGROUND

The mammalian circadian clock has evolved as an adaptation to the 24-h light/darkness cycle on earth. Maintaining cellular activities in synchrony with the activities of the organism (such as eating and sleeping) helps different tissue and organ systems coordinate and optimize their performance. The full extent of the mechanisms by which cells maintain the clock are still under investigation, but known to involve a core set of clock genes that regulate large networks of gene transcription in part by direct transcriptional activation/repression.

Chronotherapy is based on the idea that the time of day when a drug is taken is important to its efficacy. If the drug targets a clock-controlled gene that is only expressed at certain times of the day, the drug will only be effective when the target is highly expressed. This finding is important, as a recent study showed that over half (56) of the top 100 selling drugs in the United States target the product of a circadian gene. To this end, there are research and clinical trials looking into timed delivery of drugs and increasing their efficacy. For example, taking low dose aspirin for heart disease is more effective if taken at night since aspirin has a half-life of 6 hours and its target, Cox1, is upregulated at night. The timing of drugs can be applied not only to heart disease, but also to many other diseases like metabolic diseases and even brain cancers and the timing of chemotherapy.

In musculoskeletal diseases such as osteoporosis (OA) and rheumatoid arthritis (RA), there are daily inflammatory flares normally in the early morning. Cytokines present in both OA and RA knee patients exhibited diurnal expression patterns, with inflammatory cytokines peaking in the early hours of the morning. Patients with RA who took glucocorticoids at night instead of in the morning reported reduced joint pain and inflammation, showing the importance of chronotherapy in arthritis. Expanding beyond just cartilage and arthritis, cyclic delivery of parathyroid hormone (PTH) has shown improved bone growth, whereas continuous delivery of PTH showed enhanced bone resorption. Therefore, there are plenty of targets and instances where intermittent or delivery of biologics at specific times of day are required for the drug to be effective.

Unfortunately, this process can be difficult to maintain since it can require constant daily injections or delivery of drugs at inconvenient times, such as the case of arthritis where delivery of the drug in the early morning immediately before the inflammatory flare would be best.

Thus, a need exists in the art, for synthetic chronogenetic circuits driven by the circadian clock for temporal delivery of biologic drugs at specific times of day.

### SUMMARY

In an aspect, the present disclosure encompasses recombinant nucleic acid molecule comprising at least one transcriptional regulatory nucleic acid sequence of a circadian-responsive gene operably linked to a nucleic acid sequence encoding a therapeutic biologic. In some embodiments, the at least one transcriptional regulatory nucleic acid sequence is a promoter, enhancer, and/or repressor nucleic acid molecule corresponding to a promoter, repressor, and/or enhancer region of a circadian-responsive gene. In another aspect, the at least one transcriptional regulatory sequence of a circadian-responsive gene is selected from one or more of a Bmal1 gene promoter, a GSK3β gene promoter, NPAS2 gene promoter, Clocks gene promoter, Cry1 gene promoter, a Cry2 gene promoter region, a Per1 gene promoter region, a Per2 gene promoter region, a Per3 gene promoter region, Dec1 gene promoter region, a Dec2 gene promoter region, a RORα gene promoter region, RORβ gene promoter region, a REV-ERBα gene promoter region, Dbp gene promoter region, a CK1& gene promoter region, a CK1δ gene promoter region, Nfil3 gene promoter region and any combination thereof. In another embodiment, the at least one transcriptional regulatory nucleic acid sequence comprises one or more D box regions, E box regions, RRE regions and any combination thereof. In another embodiment, the at least one transcriptional regulatory region comprises one or more E-box domains which the transcription factor BMAL1 and/or CLOCK bind. In another embodiment, the transcriptional regulatory region comprises one or more PER and/or CRY repressor elements. In another embodiment, the transcriptional regulatory region comprises one or more ROR response elements. In another embodiment, the transcriptional regulatory region comprises one or more RRE elements. In another embodiment, the transcriptional regulatory region comprises one or more D-box elements upon which DBP and NFIL3 dimers bind. In another embodiment, the transcriptional regulatory region comprises, consists essentially of, or consists of the Per1 gene promoter.

In another embodiment, the transcriptional regulatory region comprises, consists essentially of, or consists of the Per2 gene promoter. In another embodiment, the transcriptional regulatory region comprises, consists essentially of, or consists of the Per3 gene promoter. In another embodiment, the transcriptional regulatory region comprises, consists essentially of, or consists of the BMAL1 gene promoter. In another embodiment, the transcriptional regulatory region comprises, consists essentially of, or consists of the CRY1 gene promoter. In another embodiment, the transcriptional regulatory region comprises, consists essentially of, or consists of the Cry2 gene promoter. In another embodiment, the transcriptional regulatory region comprises, consists essentially of, or consists of the RORA gene promoter. In another embodiment, the transcriptional regulatory region comprises, consists essentially of, or consists of the RORB gene promoter. In another embodiment, the transcriptional regulatory region comprises, consists essentially of, or consists of the REV-ERB alpha gene promoter. In another embodiment, the transcriptional regulatory region comprises, consists essentially of, or consists of the GSK3B gene promoter. In another embodiment, the transcriptional regulatory region comprises, consists essentially of, or consists of the NFIL3 gene promoter. In another embodiment, the transcriptional regulatory region comprises, consists essentially of, or consists of the CSNK1D gene promoter. In another embodiment, the transcriptional regulatory region comprises, consists essentially of, or consists of the CSNK1E gene promoter.

In each of the above embodiments, the therapeutic biologic nucleic acid sequence encodes an anti-catabolic polypeptide, an anti-inflammatory polypeptide, a pro-anabolic polypeptide, a pro-regenerative polypeptide, an anti-microbial polypeptide, an anti-pain polypeptide, a morphogen, a growth factor, an anti-cancer nucleic acid or an anti-cancer polypeptides. In one embodiment, the therapeutic biologic nucleic acid molecule encodes for one or more of IL-1Ra, sTNFR1/2, IL-10 IL-4, a growth factor from the TGFβ superfamily, IGF, CTGF, FGF, PDGF, TNF, IL-7, IL-15, IL-12, IL-2, IFN, NOS, PTGIS, Decorin, TGFβ-receptor, MMP, ALDH2, NR3C1 and any combination thereof.

In another aspect, the present disclosure provides a nucleic acid construct or vector comprising a nucleic acid molecule comprising at least one transcriptional regulatory nucleic acid sequence of a circadian-responsive gene operably linked to a nucleic acid sequence encoding a therapeutic biologic. In some embodiments, the vector is a viral vector. In some embodiments, the viral vector is an adeno-associated viral vector, a lentiviral vector or a retroviral vector.

In still another aspect, the present disclosure provides a viral particle comprising a viral vector having a nucleic acid molecule comprising at least one transcriptional regulatory nucleic acid sequence of a circadian-responsive gene operably linked to a nucleic acid sequence encoding a therapeutic biologic.

In each of the preceding aspects, the therapeutic biologic nucleic acid sequence encodes an anti-catabolic polypeptide, an anti-inflammatory polypeptide, a pro-anabolic polypeptide, a pro-regenerative polypeptide, an anti-microbial polypeptide, an anti-pain polypeptide, a morphogen, a growth factor, an anti-cancer nucleic acid or an anti-cancer polypeptides. In an exemplary embodiment, the promoter is a PTGS2 promoter and the therapeutic biologic is an IL-1 receptor antagonist.

In yet another aspect, the present disclosure provides a genetically modified cell comprising a heterologous nucleic acid sequence incorporated into its genome, wherein the heterologous nucleic acid sequence comprises a synthetic circuit having at least one transcriptional regulatory nucleic acid sequence of a circadian-responsive gene operably linked to a nucleic acid sequence encoding a therapeutic biologic.

In each of the above embodiments, the cell can be autologous or allogeneic and a somatic cell or a stem cell. In each of the above embodiments, the period, frequency, or phase of the therapeutic biologic expression is modulated through the at least one transcriptional regulatory nucleic acid molecule of a circadian responsive gene or through use of combinations of the transcriptional regulatory nucleic acid molecules of a circadian-responsive gene.

In some embodiments, the genetically modified cells is selected from an embryonic stem cells (ES), a somatic adult stem cell, a tissue-specific stem cell, an induced pluripotent stem cells (iPSCs), a progenitor cell, a fibroblast, a cardiomyocyte, a hepatocyte, an endothelial cell, a chondrocyte, a smooth muscle cell, a striated muscle cells, a bone cell, a synovial cell, a tendon cell, a ligament cell, a meniscus cell, an adipose cell, a splenocyte, an epithelial cell, a melanocyte, a neuron, an astrocyte, a microglial cell, a vascular cell, a B-cells, a dendritic cell, a natural killer cell, or a T-cell.

In another aspect, the present disclosure provides an implantable tissue comprising a genetically modified cell of the disclosure and/or a pharmaceutical composition comprising the genetically modified cell of the disclosure.

In still another aspect, the present disclosure provides a method of treating a condition, disease or disorder in a subject in need thereof, the method comprising administering an effective amount of a composition comprising a genetically modified cell of the disclosure. In some embodiments, the condition, disease or disorder is a chronic inflammatory disease, an acute inflammatory disease, a degenerative disease of any tissue or organ, chronic or acute pain, cancer, cardiovascular disease, osteoarthritis, cardiac hypertrophy, atherosclerosis, asthma, irritable bowel syndrome, muscle atrophy, angina, atrial fibrillation, hypertension, intimal hyperplasia, valve disease, scleroderma, achalasia, volvulus, diabetic nephropathy, glomerulosclerosis, cerebral edema, hydrocephalus, migraine, stroke, glaucoma, ankylosing spondylitis, carpal tunnel syndrome, chronic back pain, dupytre's contracture, osteoporosis, rheumatoid arthritis, collagenopathies, Musculo dystrophies, osteochondroplasias, polycystic kidney disease, ARDS, emphysema, pulmonary fibrosis, ventilator injury, pre-eclampsia, sexual dysfunction, and urinary incontinence.

Other aspects and iterations of the invention are described more thoroughly below.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 graphically depicts the creation of Per2-IL1Ra-t2a-Luc lentiviral circuit to deliver IL1Ra in a timed, oscillating manner.

FIG. 2 graphically depicts the in vivo regulation of Per2-IL1Ra-t2a-Luc in mice.

FIG. 3 shows Core components of the mammalian circadian clock. In the core feedback loop, the transcription factors BMAL1 (green circles) and CLOCK (blue circles) bind to E-box domains on gene promoters, including the genes for Per1 and Per2 (yellow) and Cry1 and Cry2 (red/yellow). PERs (yellow circles) and CRYs (red/yellow circles) dimerize and translocate to the nucleus after binding with casein kinase 10 (CK10) or CK16, where they repress their own transcription. The stability of PER and CRY is regulated both in the cytoplasm and within the nucleus by several proteins, including FBXL21 and FBXL3. In a second feedback loop, CLOCK and BMAL1 also regulate the transcription of genes for the nuclear receptors REV-ERBα and REV-ERBB (red circles), which compete with the retinoic acid-related orphan receptors, RORa, RORβ, and RORY (purple circles) for binding to RRE elements on the BMAL1 gene promoter, providing both positive (ROR) and negative (REV-ERB) regulation of BMAL1 transcription. A third feedback loop is mediated by CLOCK/BMAL1-mediated transcription of the gene Dbp (light blue) and the ROR/REV-ERB-mediated transcription of Nfil3 (orange). DBP (light blue circles) and NFIL3 (orange circles) dimerize and bind to D-box elements on the promoters of many of the core clock genes, providing additional layers of regulation. In addition, CLOCK/BMAL1, ROR/REV-ERB, and DBP/NFIL3 regulate the transcription of many other clock output genes (K. Cox, et al. Journal of Molecular Endocrinology, Volume 63: Issue 4, 2019).

FIG. 4 shows cell-based chronotherapy approach. The therapeutic chronogenetic circuit was created and transduced into murine induced pluripotent stem cells (miPSCs) using lentivirus. These miPSCs were then differentiated into tissue engineered cartilage. The circuit was driven by the Per2 promoter, activated by core clock component Per2 and produced both a luciferase reporter and the therapeutic drug, IL-1Ra. The Per2-IL1Ra-t2a-Luc circuit is capable of delivering an anti-inflammatory drug to target inflammatory flares.

FIG. 5A, FIG. 5B, FIG. 5C, and FIG. 5D shows characterization of chronogenetic therapy reporter circuit and tissue engineered cartilage. FIG. 5A Per2-d2eGFP-t2a-Luc bioluminescence intensity (BLI) in tissue engineered cartilage (n=3). FIG. 5A shows the mean GFP fluorescence intensity in digested pellets cast in agarose (n=5). FIG. 5A shows safranin-O staining for sGAGs (n=4/group, scale bar=100 UM). FIG. 5D shows quantification of sGAGs normalized to DNA content within each pellet (n=7/group). No significance between.

FIG. 6A, FIG. 6B, FIG. 6C, and FIG. 6D in vitro characterization of Per2-IL1Ra-t2a-Luc chronotherapy circuit. FIG. 6A BLI of Per2-IL1Ra-t2a-Luc pellets (n=3). FIG. 6B BLI of Per2-IL1Ra-t2a-Luc pellets after addition of 1 ng/ml of IL-1a (n=2). FIG. 6C Gene expression analysis of h′rn (n=6/group, fold change relative to NT control at 0 h). Samples collected every 3 hours for 72 hours. In the Per2-IL1Ra-t2a-Luc group there were oscillations of IL1rn expression and a significant decrease in expression at 27 and 39 h compared to peak expression at 69 h (p<0.05). Asterisks denotes significance compared to 69 h. FIG. 6D IL-1Ra production over the course of 3 days in the Per2-IL1Ra-t2a-Luc circuits (n=6). The chronogenetic circuit produced oscillating IL-1Ra levels with a significant decrease in expression at 42, 57, and 60 h compared to peak expression time of 63 h (p<0.01) and a significant increase in expression at peak times 33, 63, and 66 h compared to 72 h (p<0.04). Asterisks denotes significance compared to 72 h and hash denotes significance compared to 63 h

FIG. 7A, FIG. 7B, FIG. 7C, FIG. 7D, and FIG. 7E show in vivo characterization of Per2-IL1Ra-t2a-Luc chronotherapy circuit. FIG. 7A Mouse wheel-running actigraphy recorded to monitor circadian entrainment to the light cycle. Yellow background is lights on; white background is lights off. FIG. 7B BLI of Per2-IL1Ra-t2a-Luc pellets implanted in mice three days after implant (n=4 mice 16 pellets). FIG. 7C show serum IL-1Ra concentration evaluated 4 hours post BLI peak and trough expression (n=4). Asterisks denotes significance within each sample, between timepoints. FIG. 7D show BLI of pellets post entrainment to light/dark cycle (n=2 mice, 8 pellets). FIG. 7E show serum IL-1Ra concentration (n=2). Asterisks denotes significance within each sample, between timepoints.

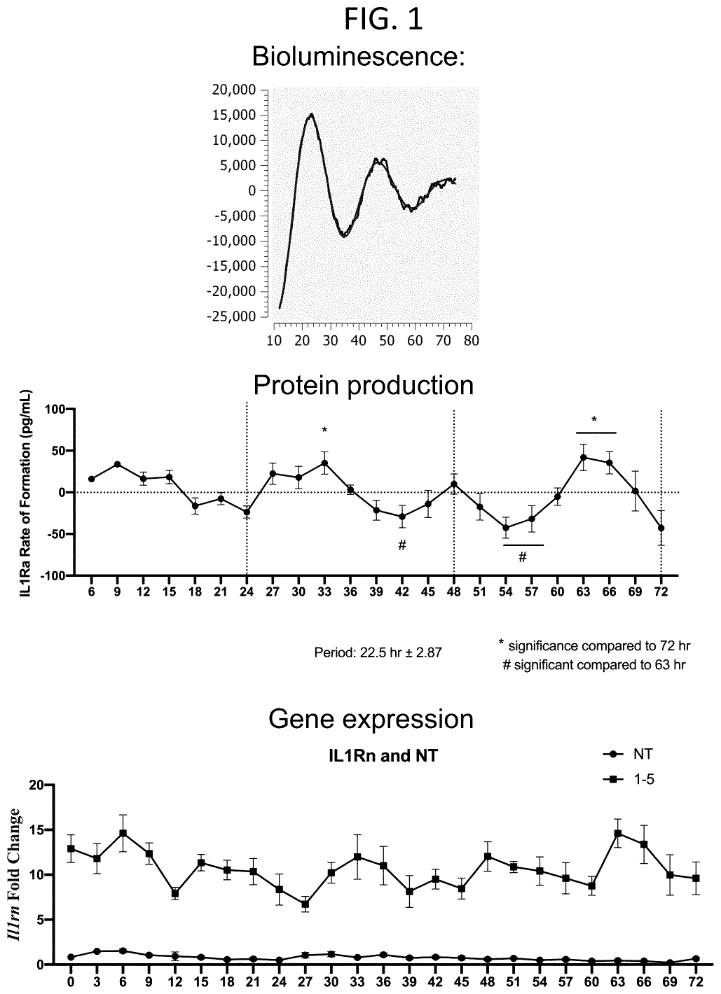

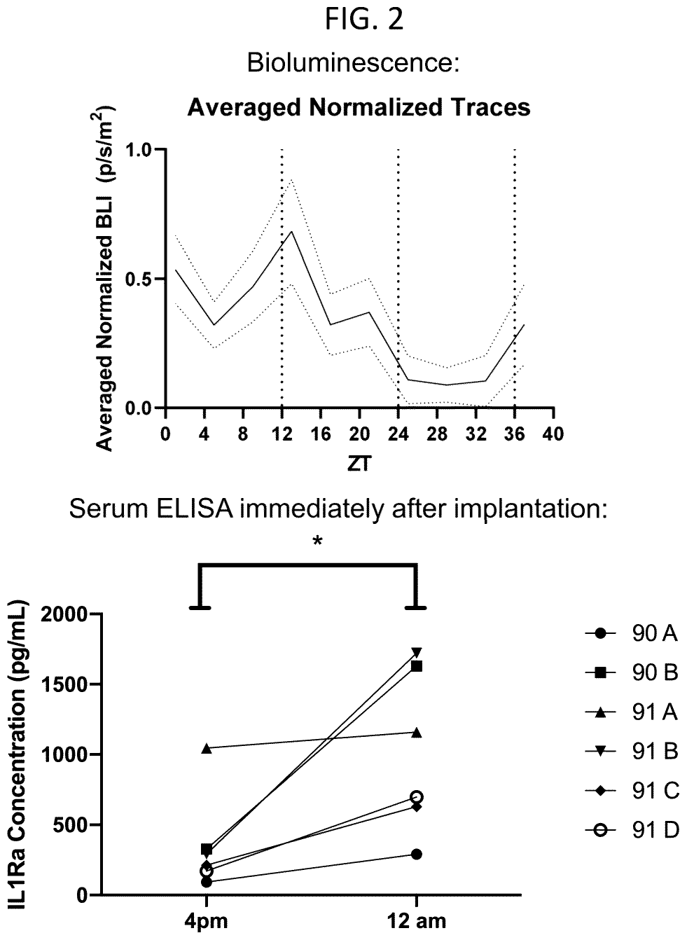

### DETAILED DESCRIPTION

The present disclosure provides compositions, systems and methods for cellular therapy comprising a genetically modified cell. In particular, a target cell is genetically modified to introduce a synthetic circuit which drives expression of a biologic therapeutic (e.g., a therapeutic polypeptide or RNA molecule). The synthetic circuit allows for activation by a prescribed input to produce the delivery of the biologic therapeutic in a controlled manner to enhance the therapeutic effect of various therapies, such as stem cell therapies, for tissue regeneration and treatment of a variety of acute and chronic diseases and cancer. This approach was motivated by the field of chronotherapy and the increase in efficacy of drugs when administered at specific times of day.

The present disclosure is based, at least in part, on the development of the first cell-based chronotherapy capable of producing an anti-inflammatory biologic at a specific time to combat the peak of inflammatory flares exhibited by subjects with chronic inflammation. In particular, the core clock gene Pert was used to drive production of a therapeutic transgene IL-1Ra in an oscillatory and timed way. This approach is the very first creation of a cell-based therapy for chronotherapy. In addition, synthetic circuits comprising the Period 2 (Per2) promoter or Bmal1 promoter driving luciferase expression have been transduced into induced pluripotent stem cells (iPSCs) and shown to have the ability to expresses luciferase in circadian oscillations. Thus, the synthetic circuits of the disclosure are tunable based on the desired phase, amplitude, and/or period of biological therapeutic expression (e.g. a transgene) based on the selection of circadian promoter regions. These synthetic circuits are then incorporated into the genome of cells which express the biological therapeutic in a controlled manner. These cells are then engineered into living tissues that are able to respond to circadian input thereby transcriptionally regulating the expression of the biologic therapeutic at specific times of day (or night) allowing to specific biological therapeutic to be most effective.

Other aspects and iterations of the invention are described more thoroughly below.

### I. Compositions

Provided herein are compositions for use in cellular therapy. The compositions including a synthetic circuit which comprises, consist essentially of, or consist of an engineered nucleic acid sequence having at least one transcriptional regulatory region (e.g. a promoter or repressor) of one or more oscillating genes (e.g., Bmal1, GSK3, NPAS2, Clocks, Cry1/2, Per1/2/3, Dec1/2, Rors (RORα/β), Rev-erbs (REV-ERBα), Dbp, CK1ε/δ, Nfil3, D box regions, E box regions, RRE regions, or any other oscillating genes downstream or upstream of these targets, including tissue specific clocks-controlled genes) operably linked to a nucleic acid sequence encoding a biologic therapeutic (e.g., a therapeutic polypeptide or RNA molecule) in a circadian mediated manner. In some embodiments, the synthetic circuit includes a regulatory region of an oscillating gene operably linked to a nucleic acid sequence encoding the biologic therapeutic. In one embodiment, the regulatory region nucleic acid molecule and the nucleic acid sequence encoding the biologic therapeutic are recombinant. In another embodiment, the regulatory region nucleic acid molecule and the nucleic acid sequence encoding the biologic therapeutic are heterologous to each other and/or to the cell in which they are incorporated. For example, the synthetic circuit can be created using a plasmid, viral transduction, or gene editing. In some embodiments, the disclosure provides the synthetic circuit incorporated into a plasmid or vector, such as an expression vector and/or viral vector. Alternatively, the regulatory region of an oscillating gene may be an endogenous nucleic acid molecule to the target cell and through targeted gene editing (e.g., CRISPR-Cas gene editing, TALENs, ZFNs, or any other method of genetic modification) the nucleic acid sequence encoding the biologic therapeutic becomes operably linked to the endogenous regulatory region of an oscillating gene such that transcriptional activation of the oscillating gene leads to expression of the therapeutic biologic in a circadian mediated manner.

The synthetic circuit of the disclosure can be regulated using the central clock system (i.e. suprachiasmatic nucleus, SCN) or a peripheral clock in the body. For example, period/frequency/phase of the expression of the therapeutic biologic via a synthetic circuit of the disclosure can be modulated through regulation of the circadian genes or through use of combinations of genes that are off-cycle from one another. In some embodiments, the period/frequency/phase of the gene oscillation can be modulated through mutation of any of the circadian genes. The overall duration and dynamics of expression of the therapeutic biologic can be controlled using a synthetic genetic counter that counts clock cycles in the cell. Methods of measuring clock cycles in a cell are known in the art and discussed in the examples below.

The genetically modified cell may be any autologous or allogeneic somatic or stem cell (e.g., adult, embryonic, or induced pluripotent stem cell). The genetically modified target cell may be endogenous or exogenous, and may be genetically modified in situ or ex vivo. Alternatively, the genetically modified target cell may be be genetically modified ex situ, and then reimplanted in the body as a cell therapy or engineered into an implantable tissue. Thus, the present disclosure provides compositions comprising the genetically modified cells.

A composition of the disclosure may optionally comprise one or more additional drug or therapeutically active agent in addition to the genetically modified cells. A composition of the disclosure may further comprise a pharmaceutically acceptable excipient, carrier, or diluent. Further, a composition of the disclosure may contain preserving agents, solubilizing agents, stabilizing agents, wetting agents, emulsifiers, salts (substances of the present invention may themselves be provided in the form of a pharmaceutically acceptable salt), buffers, coating agents, or antioxidants.

Other aspects and iterations of the invention are described more thoroughly below.

### a) Synthetic Circuit

As used herein, a “synthetic circuit” refers to a nucleic acid sequence comprising at least one transcriptional regulatory region from one or more circadian-responsive genes such that the regulatory region is operably linked to a nucleic acid sequence encoding a therapeutic biologic. As used herein, a “circadian-responsive gene” or “clock gene” refers to a reference endogenous gene that is transcriptionally regulated (completely or partially) by a cells circadian clock comprising the endogenous gene, and results in modulated expression of the coding region of the gene. Non-limiting examples of clock genes and there corresponding transcriptional regulatory nucleic acid sequences are Bmal1, GSK3β, NPAS2, Clocks, Cry1/2, Per1/2/3, Dec1/2, Rors (RORα/β), Rev-erbs (REV-ERBα), Dbp, CK1ε/δ, Nfil3, D box regions, E box regions, RRE regions and any combination thereof.

Clock genes are components of the circadian which interact with each other, in part, through their regulatory sequences in an intricate manner generating oscillations of gene expression. The underlying principle of circadian clocks is successive gene activation in the form of a cycle: the initial activation of a gene is regulated by the last one in the sequence, making up an auto-regulatory feedback loop for which one cycle takes about 24 h. Positive regulatory elements activate the expression of negative regulatory elements, which in turn stop the activity of the positive elements. This system moves away from equilibrium before returning and hence, perpetual cycling is the consequence. For example, two transcriptional activators Bmal1 (brain and muscle ARNT-like protein 1) and Clock (or Npas2 in neuronal tissue) form heterodimers in the cytoplasm and enter the nucleus where they bind to E-box sequences in the promoters of Period (Per1,2) and Cryptochrome (Cry1,2) genes contributing to the activation of their expression. In the cytoplasm various combinations of Per and Cry proteins interact with each other, enter the nucleus and inhibit the activity of Bmal1/Clock or Bmal1/Npas2 complexes. Without these complexes activating transcription of the Per and Cry genes, levels of Per and Cry transcripts and their respective protein products decline, hence Per and Cry genes shut off their own transcription.

As another example, a second loop regulates the expression of the Bmal1 gene. In the nucleus Bmal1/Clock or Bmal1/Npas2 heterodimers bind to E-boxes present in the promoters of genes that encode the retinoic acid-related orphan nuclear receptors Rev-erbα and Rorα, which compete for the ROR element (RORE) in the Bmal1 promoter. Rorα activates Bmal1 expression, while Rev-erbα represses it. As a consequence oscillations of Bmal1 and Rorα/Rev-erbα are out of phase. If activation wins over expression Bmal1 protein is produced and it forms heterodimers in the cytoplasm with Clock or Npas2 depending on the tissue. These heterodimers enter the nucleus and initiate the next cycle of gene activation of both loops.

The transcriptional activation of other clock genes are facilitated by the histone acetyl transferase (HAT) activity of the Clock protein. Histone acetylation promotes transcription through the modification of histones and allows opening of the condensed chromatin. This provides access to the transcriptional machinery. The HAT activity of Clock is necessary for the transcriptional activation of the clock genes Per and Cry and therefore seems to be essential for the generation and maintenance of endogenous circadian rhythms in mammals. Transcriptional repression is mediated by several events. Per and Cry bind to the Bmal1/Clock complex. This results in loss of HAT activity of Clock by promoting Clock phosphorylation (P) and/or inducing a conformational change of Bmal1/Clock. Loss of Clock HAT activity promotes histone deacetylation. This prevents the general transcription machinery from binding to DNA and hence transcription is repressed. Upon degradation of Per and Cry, Clock is either dephosphorylated or degraded and resynthesized. It then interacts again with Bmal1 and acetylates histones to activate a new transcription cycle.

The first clock gene, period, was discovered through investigations of Drosophila mutants with abnormal behavioral cycles. These important studies laid the foundation for understanding the molecular basis of the clock, as the per gene was found to exhibit a circadian rhythm and the PER protein, itself, was found to regulate per gene expression. Extending the studies in Drosophila, the first mammalian core clock gene, Clock, was discovered in a forward genetics screen for mice with abnormal circadian behavioral patterns. The CLOCK protein in mice has features in common with Drosophila PER, including a PAS domain (for Per, ARNT, and Sim). However, CLOCK and its binding partner, BMAL1, also have bHLH domains that allow them to bind DNA directly to regulatory elements (E-boxes) on rhythmic genes to influence their transcription.

The major targets of CLOCK/BMAL1 include other core clock genes that encode the mammalian Period ortholog (Per1, Per2, and Per3) and CRYPTOCHROME (Cry1 and Cry2) repressor proteins. These negative regulators heterodimerize and then translocate into the nucleus where they repress their own gene transcription by interacting directly with CLOCK/BMAL1. In addition to this direct transcriptional feedback, the mRNA expression of Per1/2/3 and Cry1/2 is also regulated by various mechanisms. The degradation of PER and CRY proteins is also regulated by the serine/threonine kinases, casein kinase 10 (CK10) and CK16, the F-box proteins, FBXL3 and FBXL21, and other proteins. Once negative transcriptional feedback and post-transcriptional and post-translational regulation is sufficient to decrease PER/CRY protein levels in the nucleus, repression is relieved and CLOCK/BMAL1 starts a new cycle of Per/Cry gene transcription.

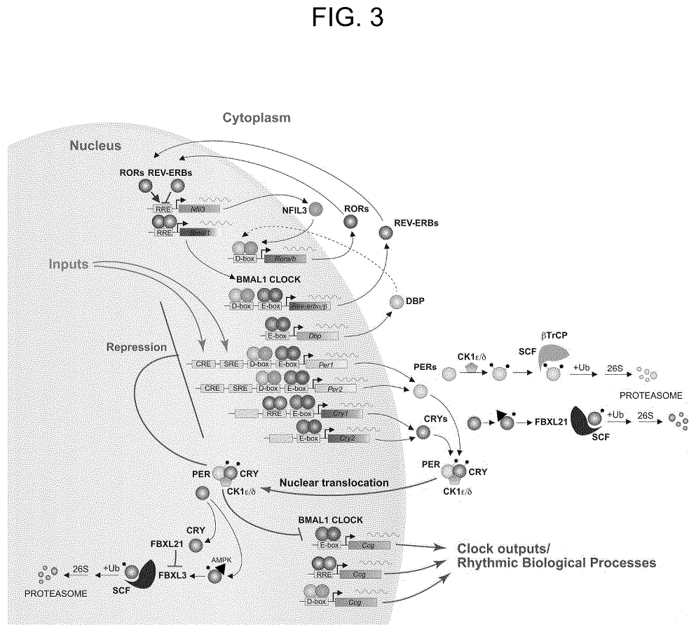

Since the initial discovery of these core mammalian clock genes, several additional genes and feedback loops have been uncovered, increasing the complexity of the mammalian circadian clock gene network (FIG. 3). In the second major transcriptional loop, CLOCK/BMAL1 activate transcription of genes for the nuclear receptors REV-ERBα and REV-ERBβ. These proteins compete with the retinoic acid-related orphan receptors RORα, RORβ, and RORγ for binding sites (ROR-binding elements) on the Bmal1 gene, providing both positive (ROR) and negative (REV-ERB) regulation of transcription, and they make an important link between the circadian clock and metabolism. A third feedback loop involves the D-box binding protein (DBP) and the nuclear factor, interleukin-3-regulated protein (NFIL3, also known as E4BP4) which are regulated by CLOCK/BMAL1 and CRY1 and bind to D-box elements on circadian promoters, including Rora and Rorb. Together, these feedback loops that make up the ‘molecular clock’ are governed by transcriptional, post-transcriptional, and post-translational regulatory mechanisms that are sufficient to maintain circadian rhythms; however, external cues are still important for synchronizing rhythms of cells within and across tissues.

Thus, the present disclosure provides a synthetic circuit comprising one or more transcriptional regulatory regions from one or more circadian-responsive genes (e.g. promoters). A “promoter” is generally understood as a nucleic acid control sequence that directs transcription of a nucleic acid. An inducible promoter is generally understood as a promoter that mediates transcription of an operably linked gene in response to a particular stimulus. A promoter can include necessary nucleic acid sequences near the start site of transcription, such as, in the case of a polymerase II type promoter, a TATA element. A promoter can optionally include distal enhancer or repressor elements, which can be located as much as several thousand base pairs from the start site of transcription. Transcriptional repressors are proteins that bind to specific sites on DNA (i.e. repressor elements) and prevent transcription of nearby genes. Enhancers are short regulatory elements of accessible DNA that help establish the transcriptional program of cells by increasing transcription of target genes. They are bound by transcription factors, co-regulators, and RNA polymerase II (RNAP II)

In one embodiment, the transcriptional regulatory region is one or more nucleic acid sequences corresponding to a promoter, repressor, and/or enhancer region of a circadian-responsive gene. In one aspect, the transcriptional regulatory region of a synthetic circuit of the disclosure comprises, consists essentially of, or consists of a E-box domains which the transcription factor BMAL1 and/or CLOCK bind. In another aspect, the transcriptional regulatory region of a synthetic circuit of the disclosure comprises, consists essentially of, or consists of one or more PER and or CRY repressor elements. In another aspect, the transcriptional regulatory region of a synthetic circuit of the disclosure comprises, consists essentially of, or consists of one or more ROR response elements. In another aspect, the transcriptional regulatory region of a synthetic circuit of the disclosure comprises, consists essentially of, or consists of one or more RRE elements. In another aspect, the transcriptional regulatory region of a synthetic circuit of the disclosure comprises, consists essentially of, or consists of one or more D-box elements upon which DBP and NFIL3 dimers bind. In another aspect, the transcriptional regulatory region of a synthetic circuit of the disclosure comprises, consists essentially of, or consists of one or more E-box domain, PER repressor element, CRY repressor element, RRE element, D-box element, ROR response element or any combination thereof.

In one embodiment, the transcriptional regulatory region nucleic acid molecule of a synthetic circuit of the disclosure has one or more of a nucleic acid sequence which comprises, consists essentially of, or consists of the Per1 gene promoter (Period Circadian Regulator 1) (e.g., a promoter as designated by GeneCard ID: GC17M010119; chr17: 8, 140,472-8, 156,506 (GRCh38/hg38); GeneHancer Identifier: GH17J008149, GH17J008142, GH17J008172, GH17J007571, GH17J007233, GH17J008220, GH17J007474, GH17J007306, GH17J007715, GH17J007434, GH17J007612, GH17J007880, GH17J008185, GH17J007853, GH17J007347, GH17J007328, GH17J008117, GH17J008093, GH17J007684, GH17J007247, GH17J008375, GH17J007279, GH17J008379, GH17J008137, GH17J008079, GH17J007176, GH17J007173, GH17J008975, GH17J008300, GH17J007450, GH17J008419, GH17J009052, GH17J008994, GH17J007175, GH17J008181, GH17J008182, GH17J008417, GH17J008412, GH17J007825, GH17J008133, GH17J008145, GH17J008143, GH17J008128, and/or GH17J008144); a nucleic acid sequence which comprises, consists essentially of, or consists of the Per2 gene promoter (Period Circadian Regulator 2) (e.g., a promoter as provided by GeneCard ID: GC02M238244; chr2: 238,244,038-238,290,102 (GRCh38/hg38); GeneHancer Identifier: GH02J238283, GH02J238299, GH02J238239, GH02J237588, GH02J237922, GH02J238369, GH02J238713, GH02J238246, GH02J237894, GH02J238247, GH02J238888, GH02J237903, GH02J238642, GH02J238294, GH02J238261, GH02J238271, GH02J238256, GH02J238279, GH02J238267, GH02J238302, GH02J238306, GH02J238304, GH02J238259, GH02J238282, GH02J238303, GH02J238260, GH02J238298, GH02J238281, GH02J238270, GH02J238309, and/or GH02J238254); a nucleic acid sequence which comprises, consists essentially of, or consists of the Per3 gene promoter (Period Circadian Regulator 3) (e.g., a promoter as designated by GeneCard ID: GH01J007782, GH01J008437, GH01J008700, GH01J007703, GH01J007297, GH01J008002, GH01J007678, GH01J007826, GH01J007993, GH01J007752, GH01J007902, GH01J007498, GH01J007912, GH01J007831, GH01J007893, GH01J008377, GH01J007722, GH01J007717, GH01J008362, GH01J007991, GH01J007755, GH01J007749, GH01J008365, GH01J007899, GH01J008036, GH01J007681, GH01J007740, GH01J008014, GH01J007761, GH01J007746, GH01J007667, GH01J007915, GH01J007921, GH01J007914, GH01J007706, GH01J007781, GH01J007810, GH01J007853, GH01J007847, GH01J007856, GH01J007885, GH01J007888, GH01J007889, GH01J007890, and/or GH01J007887); a nucleic acid sequence which comprises, consists essentially of, or consists of the BMAL1 gene promoter (aka ARNTL; Aryl Hydrocarbon Receptor Nuclear Translocator Like) (e.g., a promoter as designated by GeneCard ID: GC11P013276; chr11: 13,276,552-13,387,268 (GRCh38/hg38); GeneHancer Identifier: GH11J013276, GH11J013311, GH11J013337, GH11J013213, GH11J013258, GH11J013331, GH11J013271, GH11J013329, GH11J013324, GH11J013303, GH11J013335, GH11J013210, GH11J013250, GH11J013327, GH11J013284, GH11J013336, GH11J013221, GH11J013294, GH11J013347, GH11J013367, GH11J013306, GH11J013219, GH11J013253, GH11J013364, GH11J013228, GH11J013322, GH11J013383, GH11J013297, GH11J013239, GH11J013207, GH11J013283, GH11J013287, GH11J013288, GH11J013318, GH11J013345, GH11J013138, GH11J013461, GH11J013267, GH11J013319, GH11J013134, GH11J013117, GH11J013104, GH11J013108, GH11J013101, GH11J013114, GH11J013091, GH11J013137, GH11J013439, GH11J013159, GH11J013007, GH11J013125, GH11J013448, GH11J013097, GH11J013264, GH11J013073, GH11J013168, GH11J013064, GH11J013257, GH11J013434, GH11J013052, GH11J013227, GH11J013242, GH11J013243, GH11J013425, GH11J013144, GH11J013167, GH11J013043, GH11J013030, GH11J013436, GH11J013445, GH11J013040, GH11J013375, GH11J013150, GH11J013077, GH11J013015, GH11J013109, GH11J013443, GH11J013444, GH11J013076, GH11J013079, and/or GH11J013023); a nucleic acid sequence which comprises, consists essentially of, or consists of the CRY1 gene promoter (Cryptochrome Circadian Regulator 1) (e.g., a promoter as designated by GeneCard ID: GC12M106991; chr12: 106,991,364-107,093,872 (GRCh38/hg38); GeneHancer Identifier: GH12J107090, GH12J106130, GH12J106299, GH12J107057, GH12J107025, GH12J106581, GH12J107514, GH12J107079, GH12J106345, GH12J107109, GH12J106111, GH12J106984, GH12J107460, GH12J107903, GH12J107042, GH12J107912, GH12J107033, GH12J107014, GH12J106328, GH12J106336, GH12J107086, GH12J107121, GH12J106586, GH12J107385, GH12J107351, GH12J107948, GH12J107104, GH12J107134, GH12J107131, GH12J107126, GH12J107073, GH12J107030, GH12J107214, GH12J107200, GH12J107225, GH12J107032, GH12J106989, GH12J107236, GH12J107035, GH12J106994, GH12J107241, GH12J107076, GH12J107066, GH12J107166, GH12J107296, GH12J107055, GH12J107056, GH12J106990, GH12J107295, GH12J107287, GH12J107036, GH12J107228, GH12J107277, GH12J107178, GH12J107007, GH12J107298, GH12J107234, GH12J107307, GH12J107311, GH12J107299, GH12J107282, GH12J107302, and/or GH12J107305); a nucleic acid sequence which comprises, consists essentially of, or consists of the Cry2 gene promoter (Cryptochrome Circadian Regulator 2) (e.g., a promoter as designated by GeneCard ID: GC11P046397; chr11: 45,847,118-45,883,248 (GRCh38/hg38); GeneHancer Identifier: GH11J045846, GH11J045872, GH11J045826, GH11J045821, GH11J045818, GH11J045374, GH11J045385, GH11J045868, GH11J045842, GH11J045854, GH11J045803, GH11J045851, GH11J045879, GH11J045833, GH11J045861, GH11J045832, GH11J045875, GH11J045862, GH11J045812, and/or GH11J045865); a nucleic acid sequence which comprises, consists essentially of, or consists of the NPAS2 gene promoter (Neuronal PAS Domain Protein 2) (e.g., a promoter as designated by GeneCard ID: GC02P100820; chr2: 100,820, 139-100,996,829 (GRCh38/hg38); GeneHancer Identifier: GH02J100817, GH02J101001, GH02J100779, GH02J100741, GH02J100875, GH02J100880, GH02J100891, GH02J100921, GH02J100766, GH02J100784, GH02J100751, GH02J100730, GH02J100731, GH02J100885, GH02J100757, GH02J100857, GH02J101012, GH02J100923, GH02J101007, GH02J100853, GH02J101080, GH02J100644, GH02J100877, GH02J100594, GH02J100825, GH02J101026, GH02J100610, GH02J101047, GH02J100745, GH02J100856, GH02J100941, GH02J101163, GH02J100649, GH02J101162, GH02J101011, GH02J100949, GH02J100902, GH02J100889, GH02J100749, GH02J101160, GH02J100831, GH02J100776, GH02J100747, GH02J100861, GH02J100717, GH02J101157, GH02J101050, GH02J100823, GH02J101053, GH02J100862, GH02J100945, GH02J100726, GH02J100720, GH02J100721, GH02J100673, GH02J101161, GH02J101077, GH02J101075, GH02J100812, GH02J100796, GH02J100865, GH02J100800, GH02J100901, GH02J100845, GH02J100873, GH02J100909, GH02J100809, GH02J100844, GH02J100588, GH02J100739, GH02J100976, GH02J100984, GH02J100869, GH02J100934, GH02J100957, GH02J100706, GH02J100606, GH02J100579, GH02J100840, GH02J100980, GH02J100985, GH02J100603, GH02J100907, GH02J100992, GH02J100571, GH02J100872, GH02J100752, GH02J100690, GH02J100963, GH02J100953, GH02J100905, GH02J100703, GH02J100692, GH02J100683, GH02J100629, GH02J100982, GH02J100971, GH02J100685, GH02J100964, GH02J100592, GH02J100584, and/or GH02J100601); a nucleic acid sequence which comprises, consists essentially of, or consists of the RORA gene promoter (RAR Related Orphan Receptor A) (e.g., a promoter as designated by GeneCard ID: GC15M060488; chr15: 60,488,284-61,229,302 (GRCh38/hg38); GeneHancer Identifier: GH15J060579, GH15J061227, GH15J060525, GH15J060538, GH15J060562, GH15J060567, GH15J061835, GH15J061032, GH15J061116, GH15J061156, GH15J061173, GH15J061834, GH15J061168, GH15J061157, GH15J061167, GH15J061336, GH15J060839, GH15J061146, GH15J061198, GH15J061364, GH15J061118, GH15J060797, GH15J061141, GH15J061379, GH15J061644, GH15J060719, GH15J061186, GH15J061838, GH15J061204, GH15J061178, GH15J061182, GH15J061179, GH15J061143, GH15J061145, GH15J061155, GH15J061841, GH15J061647, GH15J060697, GH15J061212, GH15J061216, GH15J061231, GH15J061210, GH15J061084, GH15J061050, GH15J061063, GH15J061069, GH15J061010, GH15J061238, GH15J061239, GH15J061219, GH15J061112, GH15J061107, GH15J061052, GH15J061383, GH15J061038, GH15J060970, GH15J061213, GH15J061083, GH15J061088, GH15J060916, GH15J060758, GH15J061003, GH15J061079, GH15J061393, GH15J061096, GH15J060751, GH15J060913, GH15J062059, GH15J060740, GH15J060905, GH15J060899, GH15J061501, GH15J060803, GH15J060941, GH15J060795, GH15J060932, GH15J061090, GH15J061024, GH15J060648, GH15J061680, GH15J060858, GH15J060634, GH15J060760, GH15J060965, GH15J060741, GH15J061460, GH15J060718, GH15J060518, GH15J060507, GH15J060786, GH15J061528, GH15J061046, GH15J061424, GH15J061101, GH15J061028, GH15J061075, GH15J061098, GH15J060928, GH15J060778, GH15J060774, GH15J060776, GH15J060859, GH15J060773, GH15J061535, GH15J061691, GH15J060765, GH15J061702, GH15J061008, GH15J060964, GH15J060615, GH15J060604, GH15J060842, GH15J060744, GH15J061715, GH15J060727, GH15J060822, GH15J060818, GH15J060554, GH15J061907, GH15J060813, GH15J060705, GH15J060708, GH15J061748, GH15J060944, GH15J060700, GH15J060695, GH15J060523, GH15J061653, GH15J060678, GH15J060682, GH15J060673, GH15J060493, GH15J060660, GH15J060779, GH15J061073, GH15J061070, GH15J061058, GH15J061027, GH15J061438, GH15J061436, GH15J061054, GH15J061037, GH15J061017, GH15J060865, GH15J061598, GH15J060646, GH15J060772, GH15J060642, GH15J061448, GH15J060855, GH15J061537, GH15J060764, GH15J062026, GH15J060763, GH15J061697, GH15J060626, GH15J060850, GH15J060851, GH15J060849, GH15J062035, GH15J060620, GH15J061005, GH15J062046, GH15J060747, GH15J061856, GH15J060961, GH15J060999, GH15J060834, GH15J060835, GH15J060836, GH15J060909, GH15J061718, GH15J060829, GH15J061879, GH15J060731, GH15J061558, GH15J060902, GH15J061499, GH15J060994, GH15J061892, GH15J061893, GH15J060566, GH15J060821, GH15J060712, GH15J060817, GH15J061639, GH15J060993, GH15J060706, GH15J060949, GH15J061649, GH15J061648, GH15J060533, GH15J060535, GH15J060703, GH15J060701, GH15J060889, GH15J060531, GH15J060696, GH15J060694, GH15J061651, GH15J060698, GH15J061764, GH15J061515, GH15J061518, GH15J061655, GH15J060511, GH15J060681, GH15J061659, GH15J061661, GH15J060504, GH15J060875, GH15J060935, GH15J061780, GH15J061779, GH15J060494, GH15J060982, GH15J060983, GH15J060672, GH15J060490, GH15J060491, GH15J060666, GH15J060486, GH15J060783, GH15J061974, GH15J061524, GH15J060784, GH15J060656, GH15J060653, GH15J060637, GH15J060624, GH15J061829, GH15J060610, GH15J060596, GH15J060726, GH15J061890, GH15J061736, GH15J061743, GH15J061915, GH15J061923, GH15J060530, GH15J061776, GH15J060500, GH15J060485, GH15J061795, and/or GH15J060654); a nucleic acid sequence which comprises, consists essentially of, or consists of the RORB gene promoter (RAR Related Orphan Receptor B) (e.g., a promoter as designated by GeneCard ID: GC09P074497; chr9: 74,497,335-74,693, 177 (GRCh38/hg38); GeneHancer Identifier: GH09J074496, GH09J074495, GH09J074556, GH09J074333, GH09J074544, GH09J074309, GH09J074245, GH09J074459, GH09J074463, GH09J074564, GH09J074444, GH09J074304, GH09J074471, GH09J073565, GH09J074557, GH09J073548, GH09J074445, GH09J074270, GH09J074239, GH09J074250, GH09J074263, GH09J074279, GH09J074492, GH09J074501, GH09J074886, GH09J074573, GH09J074571, GH09J074379, GH09J074486, GH09J074277, GH09J074480, GH09J074578, GH09J074293, GH09J074609, GH09J074322, GH09J074802, GH09J074025, GH09J074759, GH09J074778, GH09J074777, GH09J074447, GH09J074575, GH09J074284, GH09J074855, GH09J074860, GH09J074280, GH09J073869, GH09J073845, GH09J074773, GH09J074083, GH09J074741, GH09J074644, GH09J074670, GH09J074611, GH09J074374, GH09J074630, GH09J074235, GH09J074092, GH09J074091, GH09J074085, GH09J074078, GH09J074783, GH09J074847, GH09J074326, GH09J074692, GH09J074653, GH09J074296, GH09J074308, GH09J074288, GH09J074674, GH09J074306, GH09J074690, GH09J074044, GH09J073916, GH09J074039, GH09J074869, GH09J074125, GH09J073906, GH09J074241, GH09J074036, GH09J074037, GH09J074035, GH09J074031, GH09J073891, GH09J074874, GH09J074117, GH09J073683, GH09J074272, GH09J074877, GH09J074881, GH09J074880, GH09J074884, GH09J073871, GH09J073643, GH09J073996, GH09J074825, GH09J073617, GH09J073615, GH09J073611, GH09J074087, GH09J074220, GH09J073595, GH09J073979, GH09J073577, GH09J074735, GH09J073573, GH09J073569, GH09J074217, GH09J073566, GH09J074784, GH09J073539, GH09J073515, GH09J074200, GH09J073918, GH09J073905, GH09J073680, GH09J073657, GH09J073843, GH09J073824, GH09J073791, and/or GH09J073530); a nucleic acid sequence which comprises, consists essentially of, or consists of the REV-ERB alpha gene promoter (aka NR1D1; Nuclear Receptor Subfamily 1 Group D Member 1) (e.g., a promoter as designated by GeneCard ID: GC17M040092; chr17: 40,092,793-40, 100,589 (GRCh38/hg38); GeneHancer Identifier: GH17J040102, GH17J040088, GH17J040061, GH17J040217, GH17J039183, GH17J040086, GH17J040117, GH17J040062, GH17J040083, and/or GH17J040077); a nucleic acid sequence which comprises, consists essentially of, or consists of the DBP gene promoter (D-Box Binding PAR BZIP Transcription Factor) (e.g., a promoter as designated by GeneCard ID: GC19M048630; chr19: 48,630,030-48,637,379 (GRCh38/hg38); GeneHancer Identifier: GH19J048632, GH19J048736, GH19J048897, GH19J048724, GH19J048674, GH19J048977, GH19J048768, GH19J048976, GH19J048680, GH19J048842, GH19J048764, GH19J048775, GH19J048588, GH19J048644, GH19J048622, GH19J048628, GH19J048626, and/or GH19J048621); a nucleic acid sequence which comprises, consists essentially of, or consists of the NFIL3 gene promoter (Nuclear Factor, Interleukin 3 Regulated) (e.g., a promoter as designated by GeneCard ID: GC09M091409; chr9: 91,409,045-91,425,063 (GRCh38/hg38); GeneHancer Identifier: GH09J091417, GH09J091482, GH09J091426, GH09J091191, GH09J091098, GH09J091415, GH09J091070, GH09J091391, GH09J090964, GH09J091394, GH09J091300, GH09J091085, GH09J091089, GH09J090985, GH09J091177, GH09J091126, GH09J091174, GH09J091374, GH09J091378, GH09J090979, GH09J090969, GH09J091186, GH09J091392, GH09J090987, GH09J090950, GH09J090906, GH09J091059, GH09J090769, GH09J091257, GH09J091148, GH09J091265, GH09J091150, GH09J090986, GH09J091407, GH09J091371, GH09J091067, GH09J090983, GH09J091297, GH09J091082 GH09J091416, GH09J091390, GH09J090929, GH09J090951, GH09J091071, GH09J091147, GH09J091428, GH09J091432, GH09J091427, GH09J091402, GH09J091500, GH09J091410, GH09J091406, GH09J091514, GH09J091486, GH09J091503, GH09J091494, GH09J091535, GH09J091434, GH09J091530, GH09J091484, GH09J091895, GH09J091711, GH09J091448, GH09J091560, GH09J091564, GH09J091589, GH09J091795, GH09J091884, GH09J091905, GH09J091816, GH09J091823, GH09J091837, GH09J091944, GH09J091714, GH09J091775, GH09J091856, GH09J091465, GH09J091577, GH09J091734, GH09J091681, GH09J091814, GH09J091746, GH09J091659, GH09J091720, GH09J091786, GH09J091540, GH09J091580, GH09J091546, GH09J091727, GH09J091728, GH09J091798, GH09J091802, GH09J091740, GH09J091758, GH09J091831, GH09J091834, GH09J091842, GH09J091846, GH09J091710, GH09J091715, GH09J091770, GH09J091780, GH09J091534, GH09J091572, GH09J091573, GH09J091611, GH09J091548, GH09J091558, GH09J091677, GH09J091682, GH09J091680, GH09J091890, GH09J091894, GH09J091809, GH09J091741, GH09J091812, GH09J091810, GH09J091907, GH09J091693, GH09J091690, GH09J091820, GH09J091751, GH09J091934, GH09J091935, GH09J091760, GH09J091661, GH09J091840, GH09J091772, GH09J091863, GH09J091784, GH09J091866, GH09J091873, GH09J091606, GH09J091603, GH09J091729, GH09J091875, GH09J091878, GH09J091877, GH09J091793, GH09J091643, GH09J091813, GH09J091918, GH09J091920, GH09J091925, GH09J091924, GH09J091755, GH09J091939, GH09J091765, and/or GH09J091854); a nucleic acid sequence which comprises, consists essentially of, or consists of the GSK3B gene promoter (Glycogen Synthase Kinase 3 Beta) (e.g., a promoter as designated by GeneCard ID: GC03M119821; chr3: 119,821,321-120,095,823 (GRCh38/hg38); GeneHancer Identifier: GH03J120088, GH03J120048, GH03J120067, GH03J120072, GH03J119892, GH03J120157, GH03J119809, GH03J120143, GH03J119841, GH03J120079, GH03J119973, GH03J120151, GH03J119993, GH03J120175, GH03J120057, GH03J119236, GH03J119872, GH03J120169, GH03J120101, GH03J119827, GH03J119911, GH03J120202, GH03J119821, GH03J120135, GH03J119893, GH03J120017, GH03J120035, GH03J120024, GH03J120111, GH03J120033, GH03J119825, GH03J119635, GH03J120003, GH03J120422, GH03J119831, GH03J120063, GH03J119989, GH03J119917, GH03J119548, GH03J119626, GH03J120013, GH03J120075, GH03J120031, GH03J119931, GH03J120010, GH03J119630, GH03J119950, GH03J119828, GH03J120100, GH03J120176, GH03J119742, GH03J119875, GH03J119969, GH03J120098, GH03J119627, GH03J120181, GH03J120244, GH03J120129, GH03J120214, GH03J119937, GH03J120274, GH03J119794, GH03J120198, GH03J120284, GH03J119779, GH03J120245, GH03J119944, GH03J120276, GH03J119786, GH03J120038, GH03J120036, GH03J120012, GH03J120032, GH03J120211, GH03J120227, GH03J120275, GH03J120239, GH03J120263, GH03J119977, GH03J119913, GH03J120196, GH03J120195, GH03J120229, GH03J120231, GH03J119980, GH03J119789, GH03J119788, GH03J119834, GH03J119829, GH03J119823, GH03J119914, GH03J119939, GH03J120283, GH03J119935, GH03J120272, GH03J120264, GH03J119868); a nucleic acid sequence which comprises, consists essentially of, or consists of the CSNK1E gene promoter (Casein Kinase 1 Epsilon) (e.g., a promoter as designated by GeneCard ID: GC22M055249; chr22: 38,290,691-38,318,084 (GRCh38/hg38); GeneHancer Identifier: GH22J038308, GH22J038393, GH22J038294, GH22J038289, GH22J038335, GH22J038295, GH22J038174, GH22J038768, GH22J039315, GH22J038567, GH22J037683, GH22J037847, GH22J037558, GH22J037606, GH22J038691, GH22J038422, GH22J038056, GH22J038984, GH22J038681, GH22J039232, GH22J039087, GH22J038923, GH22J037556, GH22J037400, GH22J037392, GH22J038550, GH22J038025, GH22J038217, GH22J038227, GH22J038324, GH22J038282, GH22J038353, GH22J038350, GH22J038348, GH22J038286, GH22J038309, GH22J038278, GH22J038373, GH22J038338, GH22J038274, GH22J038391, GH22J038333, GH22J038360, GH22J038390, GH22J038369, GH22J038363, GH22J038354, GH22J038394, GH22J038381, GH22J038288, GH22J038342, GH22J038387, and/or GH22J038356); a nucleic acid sequence which comprises, consists essentially of, or consists of the CSNK1D gene promoter (Casein Kinase 1 Delta) (e.g., a promoter as designated by GeneCard ID: GC17M082239; chr17: 82,239,019-82,273,750 (GRCh38/hg38); GeneHancer Identifier: GH17J082271, GH17J082291, GH17J082211, GH17J081864, GH17J081384, GH17J081709, GH17J082492, GH17J081700, GH17J082227, GH17J082696, GH17J082456, GH17J082281, GH17J082064, GH17J081348, GH17J082585, GH17J081507, GH17J082234, GH17J082671, GH17J082734, GH17J081635, GH17J081682, GH17J081976, GH17J081308, GH17J082447, GH17J081551, GH17J081990, GH17J082361, GH17J082204, GH17J082417, GH17J081664, GH17J081999, GH17J081424, GH17J082715, GH17J082590, GH17J081814, GH17J082383, GH17J081432, GH17J082553, GH17J082567, GH17J082398, GH17J082558, GH17J082544, GH17J082635, GH17J082415, GH17J082414, GH17J082565, GH17J082552, GH17J082506, GH17J082313, GH17J082381, GH17J082433, GH17J082407, GH17J082265, GH17J082332, GH17J081425, GH17J082256, GH17J082424, GH17J082175, GH17J082176, GH17J082286, GH17J082639, GH17J082477, GH17J083096, GH17J082173, GH17J082436, GH17J082547, GH17J082263, GH17J082222, GH17J082049, GH17J082246, GH17J082278, GH17J082260, GH17J082309, GH17J082225, GH17J082244, GH17J082245, GH17J082226, GH17J082276, GH17J082251, GH17J082310, GH17J082252, GH17J082284, GH17J082249, GH17J082224, GH17J082223, GH17J082277, GH17J082255, GH17J082248, and/or GH17J082312); and/or any combination thereof.

In one embodiment, a target cell is genetically modified by introducing into the genome of the cell a heterologous nucleic acid sequence comprising a regulatory nucleic acid sequence of clock gene operably linked to a nucleic acid sequence encoding a therapeutic biologic. In one aspect, the regulatory sequence of clock gene is a Bmal1 regulatory sequence, a Clocks regulatory sequence, a Cry1/2 regulatory sequence, a Per1/2/3 regulatory sequence, a Dec1/2 regulatory sequence, a Rors regulatory sequence, a Rev-erbs regulatory sequence, a Dbp regulatory sequence, or a Nfil3 regulatory sequence. In another aspect, the regulatory sequence of clock gene is from any other oscillating genes downstream or upstream of these targets, including tissue specific clocks-controlled genes. In one aspect, the regulatory sequence is a positive element, i.e., leads to increased transcription of the therapeutic biologic. In an exemplary embodiment, the positive element is a promoter or enhancer sequence (e.g., an e-box sequence). In another aspect, the regulatory sequence is a negative element, i.e., leads to decreased transcription of the therapeutic biologic. In an exemplary embodiment, the negative element is a repressor sequence. In still another aspect, the regulatory sequence includes one or more positive elements from one clock gene and one or more negative elements from a different clock gene.

In another embodiment, the target cell is genetically modified by introducing into the genome of the cell a heterologous nucleic acid sequence comprising a nucleic acid sequence encoding a therapeutic biologic. In one aspect, using a site specific nuclease the heterologous nucleic acid sequence encoding a therapeutic biologic is targeted to the genome of the target cell in a specific location such that it becomes operably linked to a regulatory sequence of clock gene. Integration of the heterologous nucleic acid may or may not disrupt expression of the endogenous corresponding clock gene. In one aspect, the regulatory sequence of clock gene is a Bmal1 regulatory sequence, a Clocks regulatory sequence, a Cry1/2 regulatory sequence, a Per1/2/3 regulatory sequence, a Dec1/2 regulatory sequence, a Rors regulatory sequence, a Rev-erbs regulatory sequence, a Dbp regulatory sequence, or a Nfil3 regulatory sequence. In another aspect, the regulatory sequence of clock gene is from any other oscillating genes downstream or upstream of these targets, including tissue specific clocks-controlled genes. In one aspect, the regulatory sequence is a positive element such as a promoter or enhancer sequence. In another aspect, the regulatory sequence is a negative element such as a repressor sequence. In still another aspect, the heterologous nucleic acid sequence may further comprise a heterologous regulatory sequence thereby creating an engineered gene circuit which regulates the expression of the therapeutic biologic through both endogenous and heterologous regulatory sequences.

In each of the above embodiments, a promoter sequence may be an ortholog, paralog, or homolog of anyone of the above referenced promoters. In one aspect, the transcriptional regulatory region is a fragment of any of the above promoter sequences. In another embodiment, the transcriptional regulatory region is any of the above promoter sequences having one or more mutations (i.e. insertion, deletions, point mutation, etc.) which modulates (e.g., increases or decreases) the phase, amplitude, or period of the biological therapeutic expression. In one embodiment, the transcriptional regulatory region is a promoter nucleic acid sequence from two or more distinct circadian-responsive genes.

In addition, the transcriptional regulatory region from a circadian-responsive gene (and combinations thereof) can be selected based on the desired temporal delivery of the biologic therapeutic. Thus, a synthetic circuit according to the present disclosure provides the use of distinct circadian signaling networks for defining the specificity, timing, and dose response for the expression of therapeutic biologic drugs. Using an engineered, living tissue construct for coordinated drug delivery obviates many of the traditional limitations of “smart” materials, such as long-term integration, rapid dynamic responses, and extended drug delivery without the need for replacement or reimplantation of the drug delivery system. The modular approach of using chronogenetic synthetic circuits provides cells within engineered tissues allows circadian clock controlled biologic drug delivery at prescribed times of day.

In some embodiments, the transcriptional regulatory region is placed upstream of a minimal promoter element and a nucleic acid sequence encoding the therapeutic biologic. For example, a TATA box derived from, in a non-limiting example, the minimal CMV promoter can be placed between the transcriptional regulatory region and therapeutic biologic such that binding of one or more transcription factors to the transcriptional regulatory region leads to transcription of the therapeutic biologic. In each of the above embodiments, additional regulatory elements can be included in the synthetic circuit as using common knowledge and methods in the art.

In one embodiment, the therapeutic biologic may include in non-limiting examples any range of endogenous or exogenous (e.g., non-mammalian) genes with therapeutic or diagnostic applications, including anti-catabolic, anti-inflammatory, pro-anabolic, pro-regenerative, pro-circadian, anti-microbial, anti-pain outputs, morphogens, growth factors, anti-cancer, etc. The proper therapeutic biologic will vary depending upon the host treated and the particular condition, disease, or disorder to be treated. In an exemplary embodiment, the therapeutic biologic is the cytokine antagonists IL-1 receptor antagonist (IL-1ra) or the type I soluble TNF receptor (STNFR1). In some embodiments, the therapeutic biologic can be, miRNA, a shRNA, a therapeutic growth factor, a transcriptional regulator, an extracellular matrix (ECM) protein, an anti-inflammatory protein, or a biomarker used to monitor treatment efficacy or disease progression. For example, the transgene may be sTNFR1, IL-1Ra, IL-4, IL6, IkB-alpha, TGFβ, IL-10, IGF, CTGF, FGF, PDGF, TNF, IL-7, IL-15, IL-12, IFN, NOS, PTGIS, Decorin, TGFb-receptor, ALDH2, NR3C1, melatonin, melatonin receptor, adensosine, adenosine receptor, a suicide gene, or a matrix degrading enzyme, such as a matrix metalloproteinase (MMP).

In one aspect, when the desired therapeutic is anti-inflammatory the therapeutic biologic nucleic acid sequence encodes for one or more of IL-1Ra, STNFR1/2, IL-10 and IL-4. In another aspect, when the desired therapeutic response is pro-anabolic, the therapeutic biologic nucleic acid sequence encodes for one or more of a growth factor from the TGFβ superfamily, IGF, CTGF, FGF, PDGF, and the like. In another aspect, when the desired therapeutic response is anti-pain, the therapeutic biologic nucleic acid sequence encodes for one or more of a kappa opioid ligand pro-peptide (prodynorphin), mu/delta opioid ligand pro-peptide (proenkephalin), delta/mu opioid ligand pro-peptide (proopiomelanocortin), and endocannabinoid ligand synthesis drivers. In another aspect, when the desired therapeutic response is anti-cancer, the therapeutic biologic nucleic acid sequence encodes for one or more of a TNF, IL-7, IL-15, IL-12, IL-2, IFN and the like. In another embodiment, when the target cell is a smooth muscle cell, the therapeutic biologic nucleic acid molecule encodes for one or more of NOS and PTGIS. In still another embodiment, when the desired therapeutic response is anti-fibrotic, the therapeutic biologic molecule encodes one or more of Decorin, TGFβ-receptor, MMPs, ALDH2 and NR3C1. In still another embodiment, when the desired response is pro-circadian the therapeutic biologic molecule encodes one or more of melatonin, melatonin receptor, adenosine, adenosine receptor, and the like.

The present disclosure provides vectors and constructs for producing genetically modified cells comprising the synthetic circuit of the disclosure. A “construct” is generally understood as any recombinant nucleic acid molecule such as a plasmid, cosmid, virus, autonomously replicating nucleic acid molecule, phage, or linear or circular single-stranded or double-stranded DNA or RNA nucleic acid molecule, derived from any source, capable of genomic integration or autonomous replication, comprising a nucleic acid molecule where one or more nucleic acid molecule has been operably linked.

A constructs of the present disclosure can contain a synthetic circuit of the disclosure operably linked to a 3′ transcription termination nucleic acid molecule. In addition, constructs can include but are not limited to additional regulatory nucleic acid molecules from, e.g., the 3′-untranslated region (3′ UTR). Constructs can include but are not limited to the 5′ untranslated regions (5′ UTR) of an mRNA nucleic acid molecule which can play an important role in translation initiation and can also be a genetic component in an expression construct. These additional upstream and downstream regulatory nucleic acid molecules may be derived from a source that is native or heterologous with respect to the other elements present on the promoter construct.

“Vector” as used herein means a nucleic acid sequence containing an origin of replication. A vector may be a viral vector, bacteriophage, bacterial artificial chromosome or yeast artificial chromosome. A vector may be a DNA or RNA vector. A vector may be a self-replicating extrachromosomal vector, and preferably, is a DNA plasmid.

Expression vector, expression construct, plasmid, or recombinant DNA construct is generally understood to refer to a nucleic acid that has been generated via human intervention, including by recombinant means or direct chemical synthesis, with a series of specified nucleic acid elements that permit transcription or translation of a particular nucleic acid in, for example, a host cell. The expression vector can be part of a plasmid, virus, or nucleic acid fragment. Typically, the expression vector can include a nucleic acid to be transcribed operably linked to a promoter.

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

In some embodiments genetically modified cells of the disclosure may be generated by cloning a nucleic acid sequence comprising a synthetic circuit of the disclosure into a viral vector. Thus, genetic modification of a target cell can be accomplished by transducing a substantially homogeneous cell composition with a recombinant DNA construct. In certain embodiments, a retroviral vector (either gamma-retroviral or lentiviral) is employed for the introduction of the DNA construct into the cell. For example, a polynucleotide comprising the regulatory sequences of the disclosure and/or a nucleic acid sequence encoding a therapeutic biologic can be cloned into a retroviral vector and expression can be driven from its endogenous promoter, from the retroviral long terminal repeat, or from a promoter specific for a target cell type of interest. Other viral vectors, or non-viral vectors may be used as well.

For initial genetic modification of a target cell, a retroviral vector is generally employed for transduction, however any other suitable viral vector or non-viral delivery system can be used. The nucleic acid sequences can be constructed with an auxiliary molecule (e.g., a cytokine) in a single, multicistronic expression cassette, in multiple expression cassettes of a single vector, or in multiple vectors. Examples of elements that create polycistronic expression cassette include, but is not limited to, various viral and non-viral Internal Ribosome Entry Sites (IRES, e.g., FGF-1 IRES, FGF-2 IRES, VEGF IRES, IGF-II IRES, NF-κB IRES, RUNX1 IRES, p53 IRES, hepatitis A IRES, hepatitis C IRES, pestivirus IRES, aphthovirus IRES, picornavirus IRES, poliovirus IRES and encephalomyocarditis virus IRES) and cleavable linkers (e.g., 2A peptides, e.g., P2A, T2A, E2A and F2A peptides). In certain embodiments, any vector disclosed herein can comprise a P2A peptide. Combinations of retroviral vector and an appropriate packaging line are also suitable, where the capsid proteins will be functional for infecting human cells. Various amphotropic virus-producing cell lines are known, including, but not limited to, PA12 (Miller, et al. (1985) Mol. Cell. Biol. 5:431-437); PA317 (Miller, et al. (1986) Mol. Cell. Biol. 6:2895-2902); and CRIP (Danos, et al. (1988) Proc. Nat. Acad. Sci. USA 85:6460-6464). Non-amphotropic particles are suitable too, e.g., particles pseudotyped with VSVG, RD114 or GALV envelope and any other known in the art.

Possible methods of transduction also include direct co-culture of the cells with producer cells, e.g., by the method of Bregni, et al. (1992) Blood 80:1418-1422, or culturing with viral supernatant alone or concentrated vector stocks with or without appropriate growth factors and polycations, e.g., by the method of Xu, et al. (1994) Exp. Hemat. 22:223-230; and Hughes, et al. (1992) J Clin. Invest. 89:1817.

Other transducing viral vectors can be used to genetically modify a target cell. In certain embodiments, the chosen vector exhibits high efficiency of infection and stable integration and expression (see, e.g., Cayouette et al., Human Gene Therapy 8:423-430, 1997; Kido et al., Current Eye Research 15:833-844, 1996; Bloomer et al., Journal of Virology 71:6641-6649, 1997; Naldini et al., Science 272:263-267, 1996; and Miyoshi et al., Proc. Natl. Acad. Sci. U.S.A. 94:10319, 1997). Other viral vectors that can be used include, for example, adenoviral, lentiviral, and adeno-associated viral vectors, vaccinia virus, a bovine papilloma virus, or a herpes virus, such as Epstein-Barr Virus (also see, for example, the vectors of Miller, Human Gene Therapy 15-14, 1990; Friedman, Science 244:1275-1281, 1989; Eglitis et al., BioTechniques 6:608-614, 1988; Tolstoshev et al., Current Opinion in Biotechnology 1:55-61, 1990; Sharp, The Lancet 337:1277-1278, 1991; Cornetta et al., Nucleic Acid Research and Molecular Biology 36:311-322, 1987; Anderson, Science 226:401-409, 1984; Moen, Blood Cells 17:407-416, 1991; Miller et al., Biotechnology 7:980-990, 1989; LeGal La Salle et al., Science 259:988-990, 1993; and Johnson, Chest 107: 77S-83S, 1995). Retroviral vectors are particularly well developed and have been used in clinical settings (Rosenberg et al., N. Engl. J. Med 323:370, 1990; Anderson et al., U.S. Pat. No. 5,399,346).

“Adeno-associated virus” or “AAV” as used interchangeably herein refers to a small virus belonging to the genus Dependovirus of the Parvoviridae family that infects humans and some other primate species. AAV is not currently known to cause disease and consequently the virus causes a very mild immune response.

Non-viral approaches can also be employed for genetic modification of a target cell. For example, a nucleic acid molecule can be introduced into a target cell by administering the nucleic acid in the presence of lipofection (Feigner et al., Proc. Natl. Acad. Sci. U.S.A. 84:7413, 1987; Ono et al., Neuroscience Letters 17:259, 1990; Brigham et al., Am. J. Med. Sci. 298:278, 1989; Staubinger et al., Methods in Enzymology 101:512, 1983), asialoorosomucoid-polylysine conjugation (Wu et al., Journal of Biological Chemistry 263:14621, 1988; Wu et al., Journal of Biological Chemistry 264:16985, 1989), or by micro-injection under surgical conditions (Wolff et al., Science 247:1465, 1990). Other non-viral means for gene transfer include transfection in vitro using calcium phosphate, DEAE dextran, electroporation, and protoplast fusion. Liposomes can also be potentially beneficial for delivery of DNA into a cell. Transplantation of normal genes into the affected tissues of a subject can also be accomplished by transferring a normal nucleic acid into a cultivatable cell type ex vivo (e.g., an autologous or heterologous primary cell or progeny thereof), after which the cell (or its descendants) are injected into a targeted tissue, are injected systemically, or incorporated into an implantable tissue which is the administered to a subject. Recombinant receptors can also be derived or obtained using transposases or targeted nucleases (e.g. Zinc finger nucleases, meganucleases, or TALE nucleases, CRISPR). Transient expression may be obtained by RNA electroporation.

Clustered regularly-interspaced short palindromic repeats (CRISPR) system is a genome editing tool discovered in prokaryotic cells. When utilized for genome editing, the system includes Cas9 (a protein able to modify DNA utilizing crRNA as its guide), CRISPR RNA (crRNA, contains the RNA used by Cas9 to guide it to the correct section of host DNA along with a region that binds to tracrRNA (generally in a hairpin loop form) forming an active complex with Cas9), trans-activating crRNA (tracrRNA, binds to crRNA and forms an active complex with Cas9), and an optional section of DNA repair template (DNA that guides the cellular repair process allowing insertion of a specific DNA sequence). CRISPR/Cas9 often employs a plasmid to transfect the target cells. The crRNA needs to be designed for each application as this is the sequence that Cas9 uses to identify and directly bind to the target DNA in a cell. The repair template carrying CAR expression cassette need also be designed for each application, as it must overlap with the sequences on either side of the cut and code for the insertion sequence. Multiple crRNA's and the tracrRNA can be packaged together to forma single-guide RNA (sgRNA). This sgRNA can be joined together with the Cas9 gene and made into a plasmid in order to be transfected into cells.

A zinc-finger nuclease (ZFN) is an artificial restriction enzyme, which is generated by combining a zinc finger DNA-binding domain with a DNA-cleavage domain. A zinc finger domain can be engineered to target specific DNA sequences which allows a zinc-finger nuclease to target desired sequences within genomes. The DNA-binding domains of individual ZFNs typically contain a plurality of individual zinc finger repeats and can each recognize a plurality of basepairs. The most common method to generate new zinc-finger domain is to combine smaller zinc-finger “modules” of known specificity. The most common cleavage domain in ZFNs is the non-specific cleavage domain from the type IIs restriction endonuclease FokI. Using the endogenous homologous recombination (HR) machinery and a homologous DNA template carrying CAR expression cassette, ZFNs can be used to insert the CAR expression cassette into genome. When the targeted sequence is cleaved by ZFNs, the HR machinery searches for homology between the damaged chromosome and the homologous DNA template, and then copies the sequence of the template between the two broken ends of the chromosome, whereby the homologous DNA template is integrated into the genome.

Transcription activator-like effector nucleases (TALEN) are restriction enzymes that can be engineered to cut specific sequences of DNA. TALEN system operates on almost the same principle as ZFNs. They are generated by combining a transcription activator-like effectors DNA-binding domain with a DNA cleavage domain. Transcription activator-like effectors (TALEs) are composed of 33-34 amino acid repeating motifs with two variable positions that have a strong recognition for specific nucleotides. By assembling arrays of these TALEs, the TALE DNA-binding domain can be engineered to bind desired DNA sequence, and thereby guide the nuclease to cut at specific locations in genome.cDNA expression for use in polynucleotide therapy methods can be directed from any suitable promoter (e.g., the human cytomegalovirus (CMV), simian virus 40 (SV40), or metallothionein promoters), and regulated by any appropriate mammalian regulatory element or intron (e.g. the elongation factor 1a enhancer/promoter/intron structure). For example, if desired, enhancers known to preferentially direct gene expression in specific cell types can be used to direct the expression of a nucleic acid. The enhancers used can include, without limitation, those that are characterized as tissue- or cell-specific enhancers. Alternatively, if a genomic clone is used as a therapeutic construct, regulation can be mediated by the cognate regulatory sequences or, if desired, by regulatory sequences derived from a heterologous source, including any of the promoters or regulatory elements described above.

The resulting cells can be grown under conditions similar to those for unmodified cells, whereby the modified cells can be expanded and used for a variety of purposes.

Any targeted genome editing methods can be used to place presently disclosed synthetic circuit at one or more endogenous gene loci of a presently disclosed target cell. For example, one or more therapeutic biologic molecules of the disclosure can be placed in the host genome such that the therapeutic biologic is operably linked to an endogenous circadian-regulated gene transcriptional element thereby creating, in one aspect, a synthetic circuit as disclosed herein. In certain embodiments, a CRISPR system is used to deliver presently disclosed synthetic circuit to one or more endogenous gene loci of a presently disclosed target cell. In certain embodiments, zinc-finger nucleases are used to deliver presently disclosed synthetic circuit to one or more endogenous gene loci of a presently disclosed target cell. In certain embodiments, a TALEN system is used to deliver presently disclosed synthetic circuit to one or more endogenous gene loci of a presently disclosed target cell.

Methods for delivering the genome editing agents/systems can vary depending on the need. In certain embodiments, the components of a selected genome editing method are delivered as DNA constructs in one or more plasmids. In certain embodiments, the components are delivered via viral vectors. Common delivery methods include but is not limited to, electroporation, microinjection, gene gun, impalefection, hydrostatic pressure, continuous infusion, sonication, magnetofection, adeno-associated viruses, envelope protein pseudotyping of viral vectors, replication-competent vectors cis and trans-acting elements, herpes simplex virus, and chemical vehicles (e.g., oligonucleotides, lipoplexes, polymersomes, polyplexes, dendrimers, inorganic Nanoparticles, and cell-penetrating peptides).

Placement of a presently disclosed synthetic circuit of the disclosure can be made at any endogenous gene locus.

### b) Genetically Modified Cells

The target cells that are used to generate the genetically modified cells are not particularly limited and can be any cell type. In non-limiting examples the target cell can be stem cells, such as embryonic stem cells (ES) and adult stem cells (somatic stem cells or tissue-specific stem cells), induced pluripotent stem cells (iPSCs), progenitor cells, fibroblasts, cardiomyocytes, hepatocytes, endothelial cells, chondrocytes, smooth or striated muscle cells, bone cells, synovial cells, tendon cells, ligament cells, meniscus cells, adipose cells, splenocytes, epithelial cells, neurons, astrocytes, microglial cells, vascular cells, B-cells, dendritic cells, natural killer cells, or T-cells. Target cells can be derived and maintained using common methods known in the art.

“Stem cells” as used herein refers to an undifferentiated cell of a multicellular organism that is capable of giving rise to indefinitely more cells of the same type, and from which certain other kinds of cell arise by differentiation. Stem cells can differentiate into specialized cells and can divide (through mitosis) to produce more stem cells. They are found in multicellular organisms. In mammals, there are two broad types of stem cells: embryonic stem cells, which are isolated from the inner cell mass of blastocysts, and adult stem cells, which are found in various tissues. In adult organisms, stem cells and progenitor cells act as a repair system for the body, replenishing adult tissues. In a developing embryo, stem cells can differentiate into all the specialized cells, such as ectoderm, endoderm and mesoderm, but also maintain the normal turnover of regenerative organs, such as blood, skin, or intestinal tissues.

“Induced pluripotent stem cells” or “iPSCs” as used interchangeably herein refers to a type of pluripotent stem cell that can be artificially derived from a non-pluripotent cell, typically an adult somatic cell, by inducing a “forced” expression of certain genes and transcription factors.

A “progenitor cell” as used herein refers to a biological cell that, like a stem cell, has a tendency to differentiate into a specific type of cell, but is already more specific than a stem cell. While stem cells can replicate indefinitely, progenitor cells can divide only a limited number of times.

(ES) cells are isolated from the inner cell mass of blastocysts of preimplantation-stage embryos. These cells require specific signals to differentiate to the desired cell type; if simply injected directly, they will differentiate into many different types of cells, resulting in a tumor derived from this abnormal pluripotent cell development (a teratoma). The directed differentiation of ES cells and avoidance of transplant rejection are just two of the hurdles that ES cell researchers still face. With their potential for unlimited expansion and pluripotency, ES cells are a potential source for regenerative medicine and tissue replacement after injury or disease.

Induced stem cells (iSC) are stem cells artificially derived from somatic, reproductive, pluripotent or other cell types by deliberate epigenetic reprogramming. They are classified as totipotent (iTC), pluripotent (iPSC) or progenitor (multipotent-iMSC, also called an induced multipotent progenitor cell-iMPC) or unipotent (iUSC) according to their developmental potential and degree of dedifferentiation.

iPSCs are somatic cells that have been genetically reprogrammed to an embryonic stem cell-like state by being forced to express genes important for maintaining the defining properties of embryonic stem cells. Although additional research is needed, iPSCs are already useful tools for drug development and modeling of diseases, and scientists hope to use them in transplantation medicine. In addition, tissues derived from iPSCs will be a nearly identical match to the cell donor and thus probably avoid rejection by the immune system. By studying iPSCs and other types of pluripotent stem cells, researchers may learn how to reprogram cells to repair damaged tissues in the human body.

Adult stem cells are undifferentiated cells, found throughout the body after development that multiply by cell division to replenish dying cells and regenerate damaged tissues. Also known as somatic stem cells, they can be found in juvenile as well as adult animals and human bodies. Scientific interest in adult stem cells is centered on their ability to divide or self-renew indefinitely, and generate all the cell types of the organ from which they originate, potentially regenerating the entire organ from a few cells. Unlike embryonic stem cells, the use of human adult stem cells in research and therapy is not considered to be controversial, as they are derived from adult tissue samples rather than human 5 day old embryos generated by IVF (in vitro fertility) clinics designated for scientific research. They have mainly been studied in humans and model organisms such as mice and rat.

The production of adult stem cells does not require the destruction of an embryo. Additionally, when adult stem cells are obtained from the intended recipient (an autograft) there is no risk of immune rejection. Adult stem cell treatments have been successfully used for many years to treat leukemia and related bone/blood cancers through bone marrow transplants.

Hematopoietic stem cells are found in the bone marrow and umbilical cord blood and give rise to all the blood cell types. Mesenchymal stem cells (MSCs) are of stromal origin and may differentiate into a variety of tissues and cell types, including: osteoblasts (bone cells), chondrocytes (cartilage cells), myocytes (muscle cells) adipocytes (fat cells). MSCs have been isolated from placenta, adipose tissue, lung, bone marrow and blood, Wharton's jelly from the umbilical cord and teeth (perivascular niche of dental pulp and periodontal ligament). MSCs are attractive for clinical therapy due to their ability to differentiate, provide trophic support, and modulate innate immune response.

Endothelial stem cells are one of the three types of multipotent stem cells found in the bone marrow. They are a rare and controversial group with the ability to differentiate into endothelial cells, the cells that line blood vessels.

Self-renewing tissues, such as the epidermis and hair follicle, continuously generate new cells to replenish the dead squames and hairs, which are sloughed into the environment. Therefore, perhaps the simplest definition of an epithelial stem cell is based on lineage: a stem cell is the cell of origin for terminally differentiated cells in adult tissues. For example, tracing the lineage of a corneocyte or hair cell back to its ultimate source in the adult skin leads to a stem cell. However, because the tools required to perform lineage analysis have not been available until recently, investigators have principally adopted definitions from the hematopoietic system. In particular, stem cells were felt to be self-renewing, multipotent, and clonogenic, similar to stem cells in the hematopoietic system that can regenerate all of the blood lineages from one cell after transplantation. In contrast to the hematopoietic stem cell field, cutaneous epithelial stem cell biologists also relied heavily on quiescence as a major stem cell characteristic.

The existence of stem cells in the adult brain has been postulated following the discovery that the process of neurogenesis, the birth of new neurons, continues into adulthood in rats. The presence of stem cells in the mature primate brain was first reported in 1967. It has since been shown that new neurons are generated in adult mice, songbirds and primates, including humans. Normally, adult neurogenesis is restricted to two areas of the brain—the subventricular zone, which lines the lateral ventricles, and the dentate gyrus of the hippocampal formation. Although the generation of new neurons in the hippocampus is well established, the presence of true self-renewing stem cells there has been debated. Under certain circumstances, such as following tissue damage in ischemia, neurogenesis can be induced in other brain regions, including the neocortex. Neural stem cells are commonly cultured in vitro as so called neurospheres—floating heterogeneous aggregates of cells, containing a large proportion of stem cells. They can be propagated for extended periods of time and differentiated into both neuronal and glia cells, and therefore behave as stem cells. However, some recent studies suggest that this behavior is induced by the culture conditions in progenitor cells, the progeny of stem cell division that normally undergo a strictly limited number of replication cycles in vivo. Furthermore, neurosphere-derived cells do not behave as stem cells when transplanted back into the brain.

Neural stem cells share many properties with hematopoietic stem cells (HSCs). Remarkably, when injected into the blood, neurosphere-derived cells differentiate into various cell types of the immune system.

Mammary stem cells provide the source of cells for growth of the mammary gland during puberty and gestation and play an important role in carcinogenesis of the breast. Mammary stem cells have been isolated from human and mouse tissue as well as from cell lines derived from the mammary gland. Single such cells can give rise to both the luminal and myoepithelial cell types of the gland, and have been shown to have the ability to regenerate the entire organ in mice.

Intestinal stem cells divide continuously throughout life and use a complex genetic program to produce the cells lining the surface of the small and large intestines. Intestinal stem cells reside near the base of the stem cell niche, called the crypts of Lieberkuhn. Intestinal stem cells are probably the source of most cancers of the small intestine and colon.

Olfactory adult stem cells have been successfully harvested from the human olfactory mucosa cells, which are found in the lining of the nose and are involved in the sense of smell. If they are given the right chemical environment these cells have the same ability as embryonic stem cells to develop into many different cell types. Olfactory stem cells hold the potential for therapeutic applications and, in contrast to neural stem cells, can be harvested with case without harm to the patient. This means they can be easily obtained from all individuals, including older patients who might be most in need of stem cell therapies.

Hair follicles contain two types of stem cells, one of which appears to represent a remnant of the stem cells of the embryonic neural crest. Similar cells have been found in the gastrointestinal tract, sciatic nerve, cardiac outflow tract and spinal and sympathetic ganglia. These cells can generate neurons, Schwann cells, myofibroblast, chondrocytes and melanocytes.

Multipotent stem cells with a claimed equivalency to embryonic stem cells have been derived from spermatogonial progenitor cells found in the testicles of laboratory mice. The extracted stem cells are known as human adult germline biggmacc stem cells (GSCs). Multipotent stem cells have also been derived from germ cells found in human testicles.

Induced stem cells (iSC) are stem cells artificially derived from somatic, reproductive, pluripotent or other cell types by deliberate epigenetic reprogramming. They are classified as totipotent (iTC), pluripotent (iPSC) or progenitor (multipotent-iMSC, also called an induced multipotent progenitor cell-iMPC) or unipotent (iUSC) according to their developmental potential and degree of dedifferentiation.

iPSCs are somatic cells that have been genetically reprogrammed to an embryonic stem cell-like state by being forced to express genes important for maintaining the defining properties of embryonic stem cells. Although additional research is needed, iPSCs are already useful tools for drug development and modeling of diseases, and scientists hope to use them in transplantation medicine. In addition, tissues derived from iPSCs will be a nearly identical match to the cell donor and thus probably avoid rejection by the immune system. By studying iPSCs and other types of pluripotent stem cells, researchers may learn how to reprogram cells to repair damaged tissues in the human body.

Chronic lung diseases such as idiopathic pulmonary fibrosis and cystic fibrosis or chronic obstructive pulmonary disease and asthma are leading causes of morbidity and mortality worldwide with a considerable human, societal, and financial burden. Several protocols have been developed for generation of the most cell types of the respiratory system, which may be useful for deriving patient-specific therapeutic cells.

Some lines of iPSCs have the potentiality to differentiate into male germ cells and oocyte-like cells in an appropriate niche (by culturing in retinoic acid and porcine follicular fluid differentiation medium or seminiferous tubule transplantation). Moreover, iPSC transplantation make a contribution to repairing the testis of infertile mice, demonstrating the potentiality of gamete derivation from iPSCs in vivo and in vitro.

T cells are a type of lymphocyte (in turn, a type of white blood cell) that play a central role in cell-mediated immunity. They can be distinguished from other lymphocytes, such as B cells and natural killer cells (NK cells), by the presence of a T-cell receptor (TCR) on the cell surface. They are called T cells because they mature in the thymus (although some also mature in the tonsils). The several subsets of T cells each have a distinct function. The majority of human T cells rearranges their alpha/beta T cell receptors and are termed alpha beta T cells and are part of adaptive immune system. Specialized gamma delta T cells, which comprise a minority of T cells in the human body (more frequent in ruminants), have invariant TCR (with limited diversity), can effectively present antigens to other T cells and are considered to be part of the innate immune system.

The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an al3T cell expressing TCR a and P chains, and a y6T cell expressing TCR y and 6 chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as 1L-2 in vivo or ex vivo. As these cells, any of cells collected from a living body, or cells obtained via ex vivo culture, for example, a T cell population obtained by the method of the present invention as it is, or obtained by freeze preservation, can be used.

Artificial T cell receptors (also known as chimeric T cell receptors, chimeric immunoreceptors, chimeric antigen receptors (CARs)) are engineered receptors, which graft an arbitrary specificity onto an immune effector cell. These receptors may be used to graft the specificity of a monoclonal antibody onto a T cell. CARs may consist of a monoclonal antibody fragment, such as a single-chain variable fragment (scFv), that presents on the outside of T-cell membranes, and is fused to intracellularly-facing stimulatory molecules. The scFv portion may recognize the tumor target. Upon binding, the intracellular stimulatory portions may initiate a signal to activate the T cell.

Artificial T cell receptors may be used as therapy for cancer using adoptive cell transfer. T cells are removed from a patient and modified so that they express receptors specific to the particular form of cancer. The T cells, which can then recognize and kill the cancer cells, are reintroduced into the patient. Modification of T-cells sourced from donors other than the patient may also be used.

The present disclosure also provides pharmaceutical compositions. The pharmaceutical composition comprises a plurality of genetically modified cells according to the present disclosure, as an active component, and at least one pharmaceutically acceptable excipient.

The pharmaceutically acceptable excipient may be a diluent, a binder, a filler, a buffering agent, a pH modifying agent, a disintegrant, a dispersant, a preservative, a lubricant, taste-masking agent, a flavoring agent, or a coloring agent. The amount and types of excipients utilized to form pharmaceutical compositions may be selected according to known principles of pharmaceutical science.

Compositions comprising the presently disclosed genetically modified cells can be conveniently provided as sterile liquid preparations, e.g., isotonic aqueous solutions, suspensions, emulsions, dispersions, or viscous compositions, which may be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol, and the like) and suitable mixtures thereof.

Sterile injectable solutions can be prepared by incorporating the genetically modified cells in the required amount of the appropriate solvent with various amounts of the other ingredients, as desired. Such compositions may be in admixture with a suitable carrier, diluent, or excipient such as sterile water, physiological saline, glucose, dextrose, or the like. The compositions can also be lyophilized. The compositions can contain auxiliary substances such as wetting, dispersing, or emulsifying agents (e.g., methylcellulose), pH buffering agents, gelling or viscosity enhancing additives, preservatives, flavoring agents, colors, and the like, depending upon the route of administration and the preparation desired. Standard texts, such as “REMINGTON'S PHARMACEUTICAL SCIENCE”, 17th edition, 1985, incorporated herein by reference, may be consulted to prepare suitable preparations, without undue experimentation.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. Prolonged absorption of the injectable pharmaceutical form can be brought about by the use of agents delaying absorption, for example, aluminum monostearate and gelatin. According to the presently disclosed subject matter, however, any vehicle, diluent, or additive used would have to be compatible with the genetically modified cells.

The compositions can be isotonic, i.e., they can have the same osmotic pressure as blood and lacrimal fluid. The desired isotonicity of the compositions may be accomplished using sodium chloride, or other pharmaceutically acceptable agents such as dextrose, boric acid, sodium tartrate, propylene glycol or other inorganic or organic solutes. Sodium chloride can be particularly for buffers containing sodium ions.

Viscosity of the compositions, if desired, can be maintained at the selected level using a pharmaceutically acceptable thickening agent. For example, methylcellulose is readily and economically available and is easy to work with. Other suitable thickening agents include, for example, xanthan gum, carboxymethyl cellulose, hydroxypropyl cellulose, carbomer, and the like. The concentration of the thickener can depend upon the agent selected. The important point is to use an amount that will achieve the selected viscosity. Obviously, the choice of suitable carriers and other additives will depend on the exact route of administration and the nature of the particular dosage form, e.g., liquid dosage form (e.g., whether the composition is to be formulated into a solution, a suspension, gel or another liquid form, such as a time release form or liquid-filled form).

The quantity of cells to be administered will vary for the subject being treated. In a one embodiment, between about 10³ and about 10¹⁰, between about 10⁵ and about 10⁹, or between about 10⁶ and about 10⁸ of the presently disclosed genetically modified cells are administered to a human subject. More effective cells may be administered in even smaller numbers. In certain embodiments, at least about 1×10⁸, about 2×10⁸, about 3×10⁸, about 4×10⁸, or about 5×10⁸ of the presently disclosed genetically modified cells are administered to a human subject. In certain embodiments, between about 1×10⁷ and 5×10⁸ of the presently disclosed genetically modified cells are administered to a human subject. The precise determination of what would be considered an effective dose may be based on factors individual to each subject, including their size, age, sex, weight, and condition of the particular subject. Dosages can be readily ascertained by those skilled in the art from this disclosure and the knowledge in the art.

The skilled artisan can readily determine the amount of cells and optional additives, vehicles, and/or carrier in compositions and to be administered in methods. Typically, any additives (in addition to the active cell(s) and/or agent(s)) are present in an amount of 0.001 to 50% (weight) solution in phosphate buffered saline, and the active ingredient is present in the order of micrograms to milligrams, such as about 0.0001 to about 5 wt %, about 0.0001 to about 1 wt %, about 0.0001 to about 0.05 wt % or about 0.001 to about 20 wt %, about 0.01 to about 10 wt %, or about 0.05 to about 5 wt %. For any composition to be administered to an animal or human, the followings can be determined: toxicity such as by determining the lethal dose (LD) and LD₅₀ in a suitable animal model e.g., rodent such as mouse; the dosage of the composition(s), concentration of components therein and timing of administering the composition(s), which elicit a suitable response. Such determinations do not require undue experimentation from the knowledge of the skilled artisan, this disclosure and the documents cited herein. And, the time for sequential administrations can be ascertained without undue experimentation.

Compositions comprising the presently disclosed genetically modified cells can be provided systemically or locally to a subject for inducing and/or enhancing a therapeutic response to a condition, disease, or disorder. In certain embodiments, the presently disclosed genetically modified cells or compositions comprising the same are directly injected into a tissue or organ of interest (e.g., an organ affected by a condition, disease or disorder). Alternatively, the presently disclosed genetically modified cells or compositions comprising the same are provided indirectly to the organ of interest, for example, by administration into the circulatory system (e.g., the organ vasculature). Expansion and differentiation agents can be provided prior to, during or after administration of the cells in vitro or in vivo.

The presently disclosed genetically modified cells can be administered in any physiologically acceptable vehicle, normally intravascularly, although they may also be introduced into bone or other convenient site where the cells may find an appropriate site for regeneration and differentiation (e.g., lymphatics). Usually, at least a population of about 1×10⁵ cells will be administered. The presently disclosed genetically modified cells can comprise a purified population of cells. Those skilled in the art can readily determine the percentage of the presently genetically modified cells in a population using various well-known methods, such as fluorescence activated cell sorting (FACS). Suitable ranges of purity in populations comprising the presently disclosed genetically modified cells are about 50% to about 55%, about 5% to about 60%, and about 65% to about 70%. In certain embodiments, the purity is about 70% to about 75%, about 75% to about 80%, or about 80% to about 85%. In certain embodiments, the purity is about 85% to about 90%, about 90% to about 95%, and about 95% to about 100%. Dosages can be readily adjusted by those skilled in the art (e.g., a decrease in purity may require an increase in dosage). The cells can be introduced by injection, catheter, or the like.

The presently disclosed compositions can be pharmaceutical compositions comprising the presently disclosed genetically modified cells and a pharmaceutically acceptable carrier. Administration can be autologous or heterologous. For example, target cells can be obtained from one subject, genetically modified according to the disclosure and administered to the same subject or a different, compatible subject. Peripheral blood derived genetically modified cells (e.g., in vivo, ex vivo or in vitro derived) can be administered via localized injection, including catheter administration, systemic injection, localized injection, intravenous injection, or parenteral administration. When administering a therapeutic composition of the presently disclosed subject matter (e.g., a pharmaceutical composition comprising a presently disclosed genetically modified cells), it can be formulated in a unit dosage injectable form (solution, suspension, emulsion).

### II. Methods

Cells disclosed herein, and/or generated using the methods disclosed herein, may be used in cellular therapy and adoptive cell transfer, for the treatment, or the manufacture of a medicament for treatment, of a variety of disorders, diseases (e.g., autoimmune diseases, inflammatory diseases), and other conditions (e.g., osteoporosis).

Thus, aspects of the present disclosure is a method for treating a subject in need thereof. The terms “treat,” “treating,” or “treatment” as used herein, refers to the provision of medical care by a trained and licensed professional to a subject in need thereof. The medical care may be a diagnostic test, a therapeutic treatment, and/or a prophylactic or preventative measure. The object of therapeutic and prophylactic treatments is to prevent or slow down (lessen) an undesired physiological change or disease/disorder. Beneficial or desired clinical results of therapeutic or prophylactic treatments include, but are not limited to, alleviation of symptoms, diminishment of extent of disease, stabilized (i.e., not worsening) state of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

Also provided is a process of treating or preventing an inflammatory or proliferative disease, disorder, or condition in a subject in need of by administration of a therapeutically effective amount of a genetically modified cells as described.

Methods described herein are generally performed on a subject in need thereof. A subject in need of the therapeutic methods described herein can be a subject having, diagnosed with, suspected of having, or at risk for developing a disease, disorder, or condition. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans or chickens. For example, the subject can be a human subject.

Generally, a safe and effective amount of genetically modified cells is, for example, that amount that would cause the desired therapeutic effect in a subject while minimizing undesired side effects. In various embodiments, an effective amount of genetically modified cells described herein can substantially inhibit progression of disease, disorder, or condition, slow the progress of disease, disorder, or condition, or limit the development of disease, disorder, or condition.

The amount of a composition described herein that can be combined with a pharmaceutically acceptable carrier to produce a single dosage form will vary depending upon the host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Toxicity and therapeutic efficacy of compositions described herein can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀, where larger therapeutic indices are generally understood in the art to be optimal.

The specific therapeutically effective dose level for any particular subject will depend upon a variety of factors including the disorder being treated and the severity of the disorder; the activity of the specific compound employed; the specific composition employed; the age, body weight, general health, sex and diet of the subject; the time of administration; the route of administration; the rate of excretion of the composition employed; the duration of the treatment; drugs used in combination or coincidental with the specific compound employed; and like factors well known in the medical arts (see e.g., Koda-Kimble et al. (2004) Applied Therapeutics: The Clinical Use of Drugs, Lippincott Williams & Wilkins, ISBN 0781748453; Winter (2003) Basic Clinical Pharmacokinetics, 4th ed., Lippincott Williams & Wilkins, ISBN 0781741475; Sharqel (2004) Applied Biopharmaceutics & Pharmacokinetics, McGraw-Hill/Appleton & Lange, ISBN 0071375503). For example, it is well within the skill of the art to start doses of the composition at levels lower than those required to achieve the desired therapeutic effect and to gradually increase the dosage until the desired effect is achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Again, each of the states, diseases, disorders, and conditions, described herein, as well as others, can benefit from compositions and methods described herein. Generally, treating a state, disease, disorder, or condition includes preventing or delaying the appearance of clinical symptoms in a mammal that may be afflicted with or predisposed to the state, disease, disorder, or condition but does not yet experience or display clinical or subclinical symptoms thereof. Treating can also include inhibiting the state, disease, disorder, or condition, e.g., arresting or reducing the development of the disease or at least one clinical or subclinical symptom thereof. Furthermore, treating can include relieving the disease, e.g., causing regression of the state, disease, disorder, or condition or at least one of its clinical or subclinical symptoms. A benefit to a subject to be treated can be either statistically significant or at least perceptible to the subject or to a physician.

Administration of the disclosed genetically modified cells can occur as a single event or over a time course of treatment. For example, genetically modified cells-based therapy can be administered daily, weekly, bi-weekly, or monthly. For more chronic conditions, treatment could extend from several weeks to several months or years.

Treatment in accord with the methods described herein can be performed prior to, concurrent with, or after conventional treatment modalities for the disease, disorder, or condition.

The genetically modified cell-therapy can be administered simultaneously or sequentially with another agent, such as an anti-inflammatory therapy, or another agent. For example, a genetically modified cell-therapy can be administered before, after, or simultaneously with another agent, such as a chemotherapeutic agent, another form of immune therapy, or radiation therapy. Simultaneous administration can occur through the administration of separate compositions, each containing one or more of a genetically modified cell-therapy and another agent, such as a chemotherapeutic agent, additional immune therapy, or radiation therapy. Simultaneous administration can occur through the administration of one composition containing two or more of genetically modified cell-therapy, an antibiotic, an anti-inflammatory, or another agent, such as a chemotherapeutic agent, immune therapy, or radiation therapy.

The administration of genetically modified cells or a population of genetically modified cells of the present disclosure be carried out by aerosol inhalation, injection, ingestion, transfusion, implantation or transplantation. The genetically modified cells, may be administered to a patient subcutaneously, intradermally, intratumorally, intranodally, intramedullary, intramuscularly, by intravenous or intralymphatic injection, or intraperitoneally. In one embodiment, the cell compositions of the present disclosure are preferably administered by intravenous injection.

As noted above, the administration of genetically modified cells or a population of genetically modified cells can consist of the administration of 10³-10⁹ cells per kg body weight, preferably 10⁵ to 10⁶ cells/kg body weight including all integer values of cell numbers within those ranges. The genetically modified cells or a population of genetically modified cells can be administrated in one or more doses. In another embodiment, the effective amount of genetically modified cells or a population of genetically modified cells are administrated as a single dose. In another embodiment, the effective amount of cells are administered as more than one dose over a period time. Timing of administration is within the judgment of a health care provider and depends on the clinical condition of the patient. The genetically modified cells or a population of genetically modified cells may be obtained from any source, such as a blood bank or a donor. While the needs of a patient vary, determination of optimal ranges of effective amounts of a given genetically modified cells population(s) for a particular disease or conditions are within the skill of the art. An effective amount means an amount which provides a therapeutic or prophylactic benefit. The dosage administered will be dependent upon the age, health and weight of the patient recipient, type of concurrent treatment, if any, frequency of treatment, and the nature of the effect desired.

The present disclosure is directed to a method of treating a subject in need thereof. The method comprises administering to subject the composition for cell therapy, as described above. The subject may have diseases include a variety of acute and chronic diseases including but not limited to genetic, degenerative, or autoimmune diseases and obesity related conditions. Diseases include acute and chronic immune and autoimmune pathologies, such as, but not limited to, rheumatoid arthritis (RA), juvenile chronic arthritis (JCA), tissue ischemia, thyroiditis, graft versus host disease (GVHD), scleroderma, diabetes mellitus, Graves' disease, disc degeneration and low back pain, allergy, acute or chronic immune disease associated with an allogenic transplantation, such as, but not limited to, renal transplantation, cardiac transplantation, bone marrow transplantation, liver transplantation, pancreatic transplantation, small intestine transplantation, lung transplantation and skin transplantation; infections, including, but not limited to, sepsis syndrome, cachexia, circulatory collapse and shock resulting from acute or chronic bacterial infection, acute and chronic parasitic and/or infectious diseases, bacterial, viral or fungal, such as a human immunodeficiency virus (HIV), acquired immunodeficiency syndrome (AIDS) (including symptoms of cachexia, autoimmune disorders, AIDS dementia complex and infections); inflammatory diseases, such as chronic inflammatory pathologies, including chronic inflammatory pathologies such as, but not limited to, sarcoidosis, chronic inflammatory bowel disease, ulcerative colitis, osteogenesis imperfecta, and Crohn's pathology or disease; vascular inflammatory pathologies, such as, but not limited to, disseminated intravascular coagulation, atherosclerosis, Kawasaki's pathology and vasculitis syndromes, such as, but not limited to, polyarteritis nodosa, Wegener's granulomatosis, Henoch-Schonlein purpura, giant cell arthritis and microscopic vasculitis of the kidneys; chronic active hepatitis; Sjogren's syndrome; spondyloarthropathies, such as ankylosing spondylitis, psoriatic arthritis and spondylitis, osteopathic arthritis and spondylitis, reactive arthritis and arthritis associated with inflammatory bowel disease; and uveitis; neurodegenerative diseases, including, but not limited to, demyelinating diseases, such as multiple sclerosis and acute transverse myelitis; myasthenia gravis; extrapyramidal and cerebellar disorders, such as lesions of the corticospinal system; disorders of the basal ganglia or cerebellar disorders; hyperkinetic movement disorders, such as Huntington's chorea and senile chorea; drug-induced movement disorders, such as those induced by drugs which block central nervous system (CNS) dopamine receptors; hypokinetic movement disorders, such as Parkinson's disease; progressive supranuclear palsy; cerebellar and spinocerebellar disorders, such as a structural lesions of the cerebellum; spinocerebellar degenerations (spinal ataxia, Friedreich's ataxia, cerebellar cortical degenerations, multiple systems degenerations (Mencel, Dejerine-Thomas, Shi-Drager, and MachadoJoseph)); and systemic disorders (Refsum's disease, abetalipoprotienemia, ataxia, telangiectasia, and mitochondrial multisystem disorder); disorders of the motor unit, such as neurogenic muscular atrophies (anterior horn cell degeneration, such as amyotrophic lateral sclerosis, infantile spinal muscular atrophy and juvenile spinal muscular atrophy); Alzheimer's disease; Down's syndrome in middle age; diffuse Lewy body disease; senile dementia of Lewy body type; Wernicke-Korsakoff syndrome; chronic alcoholism; primary biliary cirrhosis; cryptogenic fibrosing alveolitis and other fibrotic lung diseases; hemolytic anemia; Creutzfeldt-Jakob disease; subacute sclerosing panencephalitis, Hallervorden-Spatz disease; and dementia pugilistica, or any subset thereof; and malignant pathologies involving TNF-secreting tumors or other malignancies involving TNF, such as, but not limited to, leukemias (acute, chronic myelocytic, chronic lymphocytic and/or myelodyspastic syndrome); lymphomas (Hodgkin's and non-Hodgkin's lymphomas, such as malignant lymphomas (Burkitt's lymphoma or Mycosis fungoides)).

### a) Chronic Inflammatory Disease

Chronic inflammatory diseases such as arthritis are characterized by aberrant activity of cytokines such as tumor necrosis factor-a (TNF-a) and interleukin-1 (IL-1). These pro-inflammatory mediators are expressed by a wide variety of cells in musculoskeletal tissues, including myotubes, satellite cells, chondrocytes, synovial fibroblasts, osteoblasts, and resident as well as infiltrating innate immune cells. These cell types are also capable of responding to TNF-a and IL-1 a through canonical signaling via their cognate cell surface receptors. In healthy tissue, appropriate signaling of TNF-a and IL-1 contributes to organ and tissue homeostasis. In this state, these mediators promote tissue remodeling, orchestrate phagocytosis of cellular debris and immunogenic substrates, and coordinate transitions between niche stem cell quiescence and proliferation/differentiation programs. TNF-a has also been shown to enhance stem cell differentiation in a variety of efforts to enhance MSC osteogenesis. However, in chronic diseases, elevated levels of these pro-inflammatory cytokines can lead directly to pain, cytotoxicity, accelerated tissue catabolism or wasting, and exhaustion of resident stem cell niches.

A regenerative medicine approach may be used to treat chronic inflammatory diseases by generating custom-designed cells that can execute real-time, programmed responses to environmental cues, including mechanically input. Modified cells, such as stem cells, may be generated with the ability to antagonize IL-1- and TNFa-mediated inflammation in a non-limiting example. Genome-edited stem cells may be used to engineer articular cartilage tissue to establish the efficacy of therapy toward protection of tissues against cytokine-induced degeneration. This approach of repurposing mechanotransduction signaling pathways may facilitate transient production of cytokine antagonists and permit effective treatment of chronic diseases while overcoming limitations associated with delivery of large drug doses or constitutive overexpression of biologic compounds.

A therapeutic molecule may be any number of exogenous anti-cytokine therapies that effectively counteract the negative sequelae of TNF-a and IL-1 dysregulation. For example, therapeutic molecules may include competitive antagonists such as IL-1 receptor antagonist (IL-1Ra, anakinra), which alleviate symptoms of rheumatoid arthritis and the onset of post-traumatic arthritis; anti-TNF therapies, such as the soluble type 2 TNF receptor (etanercept) and monoclonal antibodies to TNF-a (adalimumab, infliximab), which have demonstrated efficacy toward offsetting pain associated with chronic and rheumatic diseases, including arthritis, ankylosing spondylitis, Crohn disease, plaque psoriasis, and ulcerative colitis; type I soluble TNFR receptor (STNFR1), which generally provided in the context of relatively high or unregulated doses.

### b) Cancer Therapy

The compositions may be used in methods of cancer therapy where the immune system is used to treat cancer. Immunotherapies fall into three main groups: cellular, antibody and cytokine. They exploit the fact that cancer cells often have subtly different molecules on their surface that can be detected by the immune system. These molecules, known as cancer antigens, are most commonly proteins, but also include molecules such as carbohydrates. Immunotherapy is used to provoke the immune system into attacking the tumor cells by using these antigens as targets.

The compositions may be used in cellular therapies, also known as cancer vaccines, usually involve the removal of immune cells from the blood or from a tumor. Immune cells specific for the tumor may be modified, cultured and returned to the patient where the immune cells attack the cancer. Cell types that can be used in this way are natural killer cells, lymphokine-activated killer cells, cytotoxic T cells and dendritic cells.

Interleukin-2 and interferon-a are examples of cytokines, proteins that regulate and coordinate the behavior of the immune system. They have the ability to enhance anti-tumor activity and thus can be used as cancer treatments. Interferon-a is used in the treatment of hairy-cell leukemia, AIDS-related Kaposi's sarcoma, follicular lymphoma, chronic myeloid leukemia and malignant melanoma. Interleukin-2 is used in the treatment of malignant melanoma and renal cell carcinoma.

Dendritic cell therapy provokes anti-tumor responses by causing dendritic cells to present tumor antigens. Dendritic cells present antigens to lymphocytes, which activates them, priming them to kill other cells that present the antigen. In cancer treatment they aid cancer antigen targeting. One method of inducing dendritic cells to present tumor antigens is by vaccination with short peptides (small parts of protein that correspond to the protein antigens on cancer cells). These peptides on their own do not stimulate a strong immune response and may be given in combination with adjuvants (highly immunogenic substances). This provokes a strong response, while also producing a (sometimes) robust anti-tumor response by the immune system. Other adjuvants include proteins or other chemicals that attract and/or activate dendritic cells, such as granulocyte macrophage colony-stimulating factor (GM-CSF). Dendritic cells can also be activated within the body (in vivo) by making tumor cells express (GM-CSF). This can be achieved by either genetically engineering tumor cells that produce GM-CSF or by infecting tumor cells with an oncolytic virus that expresses GM-CSF.

Another strategy is to remove dendritic cells from the blood of a patient and activate them outside the body (ex vivo). The dendritic cells are activated in the presence of tumor antigens, which may be a single tumor-specific peptide/protein or a tumor cell lysate (a solution of broken down tumor cells). These activated dendritic cells are put back into the body where they provoke an immune response to the cancer cells. Adjuvants are sometimes used systemically to increase the anti-tumor response provided by ex vivo activated dendritic cells. More modern dendritic cell therapies include the use of antibodies that bind to receptors on the surface of dendritic cells. Antigens can be added to the antibody and can induce the dendritic cells to mature and provide immunity to the tumor. Dendritic cell receptors such as TLR3, TLR7, TLR8 or CD40 have been used as targets by antibodies to produce immune responses.

Cytokines are a broad group of proteins produced by many types of cells present within a tumor. They have the ability to modulate immune responses. The tumor often employs it to allow it to grow and manipulate the immune response. These immune-modulating effects allow them to be used as drugs to provoke an immune response. Two commonly used groups of cytokines are interferons and interleukins.

Interferons are cytokines produced by the immune system. They are usually involved in anti-viral response, but also have use for cancer. The three groups of interferons (IFNs) are type I (IFNa and IFN (3), type II (IFNy) and type III (IFNX). IFNa has been approved for use in hairy-cell leukemia, AIDS-related Kaposi's sarcoma, follicular lymphoma, chronic myeloid leukemia and melanoma. Type I and II IFNs have been researched extensively and although both types promote anti-tumor immune system effects, only type I IFNs have been shown to be clinically effective. IFN2 shows promise for its anti-tumor effects in animal models.

Interleukins are a group of cytokines with a wide array of immune system effects. Interleukin-2 is used in the treatment of malignant melanoma and renal cell carcinoma. In normal physiology it promotes both effector T cells and T-regulatory cells, but its exact mechanism in the treatment of cancer is unknown.

### c) Regenerative Therapy Using Cell Therapy

Regenerative medicine provides the exciting potential for cell-based therapies to treat many diseases and restore damaged tissues using engineered cells for musculoskeletal applications. Modified cells derived from a myriad of adult tissues and differentiated down a lineage of choice may be tailored at the scale of the genome with application-dependent features. The compositions described here have broad applicability in regenerative medicine. For example, the ability to immobilize gene delivery vehicles capable of dictating cell fate and orchestrating ECM deposition may allow future investigators to control the spatial patterning of tissue development. This approach could indeed be applied toward engineering tissues comprised of multiple cell types and organized into regions of varied and distinct ECM constituents, a persistent challenge in the field of orthopaedic tissue engineering. Furthermore, diseases may be treated that involve complex interactions between multiple organ systems, those that drive deterioration of tissues that are not amenable to total replacement, or those in which discrimination between pathologic and healthy tissue/cells may be subtle or require real-time determination for safe and effective alleviation of disease. Such conditions may be most efficiently addressed by cells that can infiltrate, intelligently detect dysfunction, and deploy predefined therapeutic programs to resolve anomalous behavior of endogenous cells or ECM disorganization/degeneration. Employing these and other tools from synthetic biology together under the auspices of a functional cellular and tissue engineering paradigm, which aims to fully characterize and recapitulate features critical for successful cell/tissue replacement, will likely serve to advance the field of regenerative medicine toward the establishment of clinically effective therapies for a host of diseases.

In some embodiments, the site-specific nucleases may be used to generate functional deficiencies or complete knock-out of the proteins coded by the targeted genes in human iPSCs. Genetically modified iPSCs may be differentiated into chondrocytes using established techniques. Feedback-controlled gene circuits may be designed to modulate the production of soluble TNF receptors—specifically soluble TNF receptor 1 (sTNFR1), which blocks TNF signaling—in response to dynamic TNF levels. In some embodiments, this process may be performed in induced pluripotent stem cells (iPSCs), which can be expanded indefinitely, thus facilitating the complex genetic manipulations required for genome editing. To produce an implant with long-term in vivo stability, the rewired iPSCs may be differentiated into cartilage cells (chondrocytes), a robust, non-migratory cell that naturally responds to TNF. These cells may be formed into a tissue-engineered cartilage implant that can be implanted in the joint to repair damaged cartilage or subcutaneously to provide self-regulated, systemic anti-TNF.

### d) Osteoarthritis

The modified cells may be used in musculoskeletal regenerative medicine applications, such as developing therapies for osteoarthritis. Osteoarthritis (OA) is a progressive disease of synovial joints characterized by the destruction of articular cartilage. Surgical treatment options for focal cartilage defects include arthroscopic debridement, marrow stimulation via microfracture, and autologous transplantation of host tissue or ex vivo expanded autologous chondrocytes. Most of these surgical options lead to the development of a fibrocartilage matrix that serves only as a temporary solution to a complex and demanding biomechanical problem. For larger defects, joint arthroplasty serves as the most promising treatment option. While effective at restoring function to the joint, the need to revise an increasing number of primary arthroplasties means that a more functional, long-term solution is needed.

Inflammation plays a key role in the pathogenesis and progression of osteoarthritis (OA) and may compromise engineered tissue substitutes. Chondrocytes and synovial fibroblasts in OA joints are subjected to increased interleukin (IL)-1, IL-6, IL-17 and tumor necrosis factor (TNF)-a signaling. The activity of these cytokines in OA joints leads to increased production of matrix metalloproteinases (MMPs), aggrecanases, inducible nitric oxide synthase, and prostaglandin E2. These and other factors ultimately lead to suppression of cartilage-specific genes such as COL2A1, downregulation of proteoglycan levels, degeneration of extracellular matrix, and chondrocyte apoptosis. Furthermore, prolonged inflammatory signaling mediated by IL-la inhibits chondrogenic induction of stem cells and results in degradation of stem cell derived cartilage. The pro-inflammatory environment of the OA joint therefore necessitates a tissue substitute designed to resist inflammation-mediated degradation.

### e) Pain

Pain is generally defined as an unpleasant sensory and emotional experience associated with actual or potential tissue damage or described in terms of such damage. Merskey H, Bogduk N, eds., Classification of Chronic Pain, International Association for the Study of Pain (IASP) Task Force on Taxonomy, IASP Press: Seattle, 209-214, 1994. Because the perception of pain is highly subjective, it is one of the most difficult pathologies to diagnose and treat effectively.

In one aspect, provided herein is a method of treating an individual having pain, comprising administering to the individual a therapeutically effective amount of a composition comprising a genetically modified cell of the disclosure, wherein the therapeutically effective amount is an amount sufficient to cause a detectable improvement in said pain or a symptom associated with said pain. In one embodiment, said method additionally comprises determining a first level of pain in said individual prior to administration of said genetically modified cells, and determining a second level of pain in said individual after administration of said genetically modified cells, wherein said therapeutically effective amount of genetically modified cells reduces said second level of said pain as compared to said first level of pain.

In certain embodiments, the therapeutically effective amount of genetically modified cells, when administered, results in greater, or more long-lasting, improvement of pain in the individual as compared to administration of a placebo.

In certain embodiments, the pain is nociceptive pain. Nociceptive pain is typically elicited when noxious stimuli such as inflammatory chemical mediators are released following tissue injury, disease, or inflammation and are detected by normally functioning sensory receptors (nociceptors) at the site of injury. See, e.g., Koltzenburg, M. Clin. J. of Pain 16: S131-S138 (2000). Examples of causes of nociceptive pain include, but are not limited to, chemical or thermal burns, cuts and contusions of the skin, osteoarthritis, rheumatoid arthritis, tendonitis, and myofascial pain. In certain embodiments, nociceptive pain is stimulated by inflammation.

### III. Kits

Also provided are kits. Such kits can include an agent or composition described herein and, in certain embodiments, instructions for administration. Such kits can facilitate performance of the methods described herein. When supplied as a kit, the different components of the composition can be packaged in separate containers and admixed immediately before use. Components include, but are not limited to genetically modified cells of the disclosure or a nucleic acid sequence comprising a synthetic circuit of the disclosure, and delivery systems. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components.

Kits may also include reagents in separate containers such as, for example, sterile water or saline to be added to a lyophilized active component packaged separately. For example, sealed glass ampules may contain a lyophilized component and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium or video. Detailed instructions may not be physically associated with the kit; instead, a user may be directed to an Internet web site specified by the manufacturer or distributor of the kit.

A control sample or a reference sample as described herein can be a sample from a healthy subject or from a randomized group of subjects. A reference value can be used in place of a control or reference sample, which was previously obtained from a healthy subject or a group of healthy subject. A control sample or a reference sample can also be a sample with a known amount of a detectable compound or a spiked sample.

The methods and algorithms of the invention may be enclosed in a controller or processor. Furthermore, methods and algorithms of the present invention, can be embodied as a computer implemented method or methods for performing such computer-implemented method or methods, and can also be embodied in the form of a tangible or non-transitory computer readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer program include, for example, floppy disks and diskettes, compact disk (CD)-ROMs (whether or not writeable), DVD digital disks, RAM and ROM memories, computer hard drives and back-up drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All references, patents and patent applications disclosed herein are incorporated by reference with respect to the subject matter for which each is cited, which in some cases may encompass the entirety of the document.

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

The term “about,” as used herein, refers to variation of in the numerical quantity that can occur, for example, through typical measuring techniques and equipment, with respect to any quantifiable variable, including, but not limited to, mass, volume, time, distance, and amount. Further, given solid and liquid handling procedures used in the real world, there is certain inadvertent error and variation that is likely through differences in the manufacture, source, or purity of the ingredients used to make the compositions or carry out the methods and the like. The term “about” also encompasses these variations, which can be up to ±5%, but can also be ±4%, 3%, 2%, 1%, etc. Whether or not modified by the term “about,” the claims include equivalents to the quantities.

When introducing elements of the present disclosure or the preferred aspects(s) thereof, the articles “a,” “an,” “the,” and “said” are intended to mean that there are one or more of the elements. The terms “comprising,” “including,” and “having” are intended to be inclusive and mean that there may be additional elements other than the listed elements.

The terms “patient,” “subject,” “individual,” and the like are used interchangeably herein, and refer to any animal or cells thereof whether in vitro or in situ, amenable to the methods described herein. In certain non-limiting embodiments, the patient, subject or individual is a human.

As used herein, the term “subject” refers to a mammal, preferably a human. The mammals include, but are not limited to, humans, primates, livestock, rodents, and companion animals. A subject may be waiting for medical care or treatment, may be under medical care or treatment, or may have received medical care or treatment.

The following definitions and methods are provided to better define the present invention and to guide those of ordinary skill in the art in the practice of the present invention. Unless otherwise noted, terms are to be understood according to conventional usage by those of ordinary skill in the relevant art.

The terms “heterologous DNA sequence”, “exogenous DNA segment” or “heterologous nucleic acid,” as used herein, each refer to a sequence that originates from a source foreign to the particular host (target) cell or, if from the same source, is modified from its original form. Thus, a heterologous gene in a host cell includes a gene that is endogenous to the particular host cell but has been modified through, for example, the use of DNA shuffling or cloning. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides. A “homologous” DNA sequence is a DNA sequence that is naturally associated with a host cell into which it is introduced.

A “transcribable nucleic acid molecule” as used herein refers to any nucleic acid molecule capable of being transcribed into an RNA molecule. Methods are known for introducing constructs into a cell in such a manner that the transcribable nucleic acid molecule is transcribed into a functional mRNA molecule that is translated and therefore expressed as a protein product. Constructs may also be constructed to be capable of expressing antisense RNA molecules, in order to inhibit translation of a specific RNA molecule of interest. For the practice of the present disclosure, conventional compositions and methods for preparing and using constructs and host cells are well known to one skilled in the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754).

The “transcription start site” or “initiation site” is the position surrounding the first nucleotide that is part of the transcribed sequence, which is also defined as position +1. With respect to this site, all other sequences of the gene and its controlling regions can be numbered. Downstream sequences (i.e., further protein encoding sequences in the 3′ direction) can be denominated positive, while upstream sequences (mostly of the controlling regions in the 5′ direction) are denominated negative.

As used herein the term “gene” refers to a polynucleotide (e.g., a DNA segment) that encodes a polypeptide and includes regions preceding and following the coding regions as well as intervening sequences (introns) between individual coding segments (exons). The regions preceding and following the coding regions may comprise regulatory nucleotide sequences such that they are operably linked to the coding regions. “Operably-linked” or “functionally linked” refers preferably to the association of nucleic acid sequences on a single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. The two nucleic acid molecules may be part of a single contiguous nucleic acid molecule and may be adjacent. For example, a promoter is operably linked to a gene of interest if the promoter regulates or mediates transcription of the gene of interest in a cell.

Nucleic acid or amino acid sequences are “operably linked” (or “operatively linked”) when placed into a functional relationship with one another. For instance, a promoter or enhancer is operably linked to a coding sequence if it regulates, or contributes to the modulation of, the transcription of the coding sequence. Operably linked DNA sequences are typically contiguous, and operably linked amino acid sequences are typically contiguous and in the same reading frame. However, since enhancers generally function when separated from the promoter by up to several kilobases or more and intronic sequences may be of variable lengths, some polynucleotide elements may be operably linked but not contiguous. Similarly, certain amino acid sequences that are non-contiguous in a primary polypeptide sequence may nonetheless be operably linked due to, for example folding of a polypeptide chain. With respect to fusion polypeptides, the terms “operatively linked” and “operably linked” can refer to the fact that each of the components performs the same function in linkage to the other component as it would if it were not so linked.

“Transformed,” “transgenic,” and “recombinant” refer to a host cell or organism such as a bacterium, cyanobacterium, animal, or a plant into which a heterologous nucleic acid molecule has been introduced. The nucleic acid molecule can be stably integrated into the genome as generally known in the art and disclosed (Sambrook 1989; Innis 1995; Gelfand 1995; Innis & Gelfand 1999). Known methods of PCR include, but are not limited to, methods using paired primers, nested primers, single specific primers, degenerate primers, gene-specific primers, vector-specific primers, partially mismatched primers, and the like. The term “untransformed” refers to normal cells that have not been through the transformation process.

Design, generation, and testing of the variant nucleotides, and their encoded polypeptides, having the above-required percent identities and retaining a required activity of the expressed protein is within the skill of the art. For example, directed evolution and rapid isolation of mutants can be according to methods described in references including, but not limited to, Link et al. (2007) Nature Reviews 5 (9), 680-688; Sanger et al. (1991) Gene 97 (1), 119-123; Ghadessy et al. (2001) Proc Natl Acad Sci USA 98 (8) 4552-4557. Thus, one skilled in the art could generate a large number of nucleotide and/or polypeptide variants having, for example, at least 50-99% identity to the reference sequence described herein and screen such for desired phenotypes according to methods routine in the art.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

“Donor DNA”, “donor template” and “repair template” as used interchangeably herein refers to a double-stranded DNA fragment or molecule that includes at least a portion of the gene of interest. The donor DNA may encode a full-functional protein or a partially-functional protein.

“Endogenous gene” as used herein refers to a gene that originates from within an organism, tissue, or cell. An endogenous gene is native to a cell, which is in its normal genomic and chromatin context, and which is not heterologous to the cell. Such cellular genes include, e.g., animal genes, plant genes, bacterial genes, protozoal genes, fungal genes, mitochondrial genes, and chloroplastic genes.

“Functional” and “full-functional” as used herein describes protein that has biological activity. A “functional gene” refers to a gene transcribed to mRNA, which is translated to a functional protein.

“Genetic construct” as used herein refers to the DNA or RNA molecules that comprise a nucleotide sequence that encodes a protein. The coding sequence includes initiation and termination signals operably linked to regulatory elements including a promoter and polyadenylation signal capable of directing expression in the cells of the individual to whom the nucleic acid molecule is administered. As used herein, the term “expressible form” refers to gene constructs that contain the necessary regulatory elements operably linked to a coding sequence that encodes a protein such that when present in the cell of the individual, the coding sequence will be expressed.

“Genome editing” as used herein refers to changing the endogenous DNA of a cell. Genome editing may include the addition of nucleic acids, deletion of nucleic acids, or restoring a mutant gene. Genome editing may include knocking out a gene, such as a mutant gene or a normal gene, or knocking-in a heterologous gene or protein encoding region thereof.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (naturally occurring) form of the cell or express a second copy of a native gene that is otherwise normally or abnormally expressed, under expressed or not expressed at all.

“Homology-directed repair” or “HDR” as used interchangeably herein refers to a mechanism in cells to repair double strand DNA lesions when a homologous piece of DNA is present in the nucleus, mostly in G2 and S phase of the cell cycle. HDR uses a donor DNA template to guide repair and may be used to create specific sequence changes to the genome, including the targeted addition of whole genes. If a donor template is provided along with the site specific nuclease, such as with a CRISPR/Cas9-based systems, then the cellular machinery may repair the break by homologous recombination, which is enhanced several orders of magnitude in the presence of DNA cleavage. When the homologous DNA piece is absent, non-homologous end joining may take place instead.

“Non-homologous end joining (NHEJ) pathway” as used herein refers to a pathway that repairs double-strand breaks in DNA by directly ligating the break ends without the need for a homologous template. The template-independent re-ligation of DNA ends by NHEJ is a stochastic, error-prone repair process that introduces random micro-insertions and micro-deletions (indels) at the DNA breakpoint. This method may be used to intentionally disrupt, delete, or alter the reading frame of targeted gene sequences. NHEJ typically uses short homologous DNA sequences called microhomologies to guide repair. These microhomologies are often present in single-stranded overhangs on the end of double-strand breaks. When the overhangs are perfectly compatible, NHEJ usually repairs the break accurately, yet imprecise repair leading to loss of nucleotides may also occur, but is much more common when the overhangs are not compatible.

“Nuclease mediated NHEJ” as used herein refers to NHEJ that is initiated after a nuclease, such as a Cas9, cuts double stranded DNA.

“Site-specific nuclease” as used herein refers to an enzyme capable of specifically recognizing and cleaving DNA sequences. The site-specific nuclease may be engineered. Examples of engineered site-specific nucleases include zinc finger nucleases (ZFNs), TAL effector nucleases (TALENs), and CRISPR/Cas-based systems.

“Target region” as used herein refers to the region of the target gene to which the site-specific nuclease is designed to bind and cleave.

“Nucleic acid” or “oligonucleotide” or “polynucleotide” as used herein means at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. A single strand provides a probe that may hybridize to a target sequence under stringent hybridization conditions. Thus, a nucleic acid also encompasses a probe that hybridizes under stringent hybridization conditions.

Nucleic acids may be single stranded or double stranded, or may contain portions of both double stranded and single stranded sequence. The nucleic acid may be DNA, both genomic and cDNA, RNA, or a hybrid, where the nucleic acid may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods.

Nucleotide and/or amino acid sequence identity percent (%) is understood as the percentage of nucleotide or amino acid residues that are identical with nucleotide or amino acid residues in a candidate sequence in comparison to a reference sequence when the two sequences are aligned. To determine percent identity, sequences are aligned and if necessary, gaps are introduced to achieve the maximum percent sequence identity. Sequence alignment procedures to determine percent identity are well known to those of skill in the art. Often publicly available computer software such as BLAST, BLAST2, ALIGN2, or Megalign (DNASTAR) software is used to align sequences. Those skilled in the art can determine appropriate parameters for measuring alignment, including any algorithms needed to achieve maximal alignment over the full-length of the sequences being compared. When sequences are aligned, the percent sequence identity of a given sequence A to, with, or against a given sequence B (which can alternatively be phrased as a given sequence A that has or comprises a certain percent sequence identity to, with, or against a given sequence B) can be calculated as: percent sequence identity=X/Y100, where X is the number of residues scored as identical matches by the sequence alignment program's or algorithm's alignment of A and B and Y is the total number of residues in B. If the length of sequence A is not equal to the length of sequence B, the percent sequence identity of A to B will not equal the percent sequence identity of B to A.

Generally, conservative substitutions can be made at any position so long as the required activity is retained. So-called conservative exchanges can be carried out in which the amino acid which is replaced has a similar property as the original amino acid, for example, the exchange of Glu by Asp, Gln by Asn, Val by Ile, Leu by Ile, and Ser by Thr. For example, amino acids with similar properties can be Aliphatic amino acids (e.g., Glycine, Alanine, Valine, Leucine, Isoleucine); Hydroxyl or sulfur/selenium-containing amino acids (e.g., Serine, Cysteine, Selenocysteine, Threonine, Methionine); Cyclic amino acids (e.g., Proline); Aromatic amino acids (e.g., Phenylalanine, Tyrosine, Tryptophan); Basic amino acids (e.g., Histidine, Lysine, Arginine); or Acidic and their Amide (e.g., Aspartate, Glutamate, Asparagine, Glutamine). Deletion is the replacement of an amino acid by a direct bond. Positions for deletions include the termini of a polypeptide and linkages between individual protein domains. Insertions are introductions of amino acids into the polypeptide chain, a direct bond formally being replaced by one or more amino acids. An amino acid sequence can be modulated with the help of art-known computer simulation programs that can produce a polypeptide with, for example, improved activity or altered regulation. On the basis of this artificially generated polypeptide sequences, a corresponding nucleic acid molecule coding for such a modulated polypeptide can be synthesized in-vitro using the specific codon-usage of the desired host cell.

“Highly stringent hybridization conditions” are defined as hybridization at 65° C. in a 6×SSC buffer (i.e., 0.9 M sodium chloride and 0.09 M sodium citrate). Given these conditions, a determination can be made as to whether a given set of sequences will hybridize by calculating the melting temperature (Tm) of a DNA duplex between the two sequences. If a particular duplex has a melting temperature lower than 65° C. in the salt conditions of a 6×SSC, then the two sequences will not hybridize. On the other hand, if the melting temperature is above 65° C. in the same salt conditions, then the sequences will hybridize. In general, the melting temperature for any hybridized DNA: DNA sequence can be determined using the following formula: Tm=81.5° C.+16.6 (log 10 [Na+])+0.41 (fraction G/C content)−0.63 (% formamide)−(600/l). Furthermore, the Tm of a DNA: DNA hybrid is decreased by 1-1.5° C. for every 1% decrease in nucleotide identity (see e.g., Sambrook and Russel, 2006).

Host cells can be transformed using a variety of standard techniques known to the art (see e.g., Sambrook and Russel (2006) Condensed Protocols from Molecular Cloning: A Laboratory Manual, Cold Spring Harbor Laboratory Press, ISBN-10:0879697717; Ausubel et al. (2002) Short Protocols in Molecular Biology, 5th ed., Current Protocols, ISBN-10:0471250929; Sambrook and Russel (2001) Molecular Cloning: A Laboratory Manual, 3d ed., Cold Spring Harbor Laboratory Press, ISBN-10:0879695773; Elhai, J. and Wolk, C. P. 1988. Methods in Enzymology 167, 747-754). Such techniques include, but are not limited to, viral infection, calcium phosphate transfection, liposome-mediated transfection, microprojectile-mediated delivery, receptor-mediated uptake, cell fusion, electroporation, and the like. The transfected cells can be selected and propagated to provide recombinant host cells that comprise the expression vector stably integrated in the host cell genome.

Exemplary nucleic acids which may be introduced to a host cell include, for example, DNA sequences or genes from another species, or even genes or sequences which originate with or are present in the same species, but are incorporated into recipient cells by genetic engineering methods. The term “exogenous” is also intended to refer to genes that are not normally present in the cell being transformed, or perhaps simply not present in the form, structure, etc., as found in the transforming DNA segment or gene, or genes which are normally present and that one desires to express in a manner that differs from the natural expression pattern, e.g., to over-express. Thus, the term “exogenous” gene or DNA is intended to refer to any gene or DNA segment that is introduced into a recipient cell, regardless of whether a similar gene may already be present in such a cell. The type of DNA included in the exogenous DNA can include DNA which is already present in the cell, DNA from another individual of the same type of organism, DNA from a different organism, or a DNA generated externally, such as a DNA sequence containing an antisense message of a gene, or a DNA sequence encoding a synthetic or modified version of a gene.

Host strains developed according to the approaches described herein can be evaluated by a number of means known in the art (see e.g., Studier (2005) Protein Expr Purif. 41 (1), 207-234; Gellissen, ed. (2005) Production of Recombinant Proteins: Novel Microbial and Eukaryotic Expression Systems, Wiley-VCH, ISBN-10:3527310363; Baneyx (2004) Protein Expression Technologies, Taylor & Francis, ISBN-10:0954523253).

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

“Cell therapy”, “cellular therapy”, or “cytotherapy” as used herein refers to a therapy in which cellular material is administered into a patient or one or more cells in a subject are genetically modified in accordance with the present disclosure. The cellular material may be intact, living cells and provide a therapeutic response to a condition, disease, or disorder in the subject by reducing or preventing one or more symptoms associated with the condition, disease, or disorder; or reduces or prevents progression of the condition, disease, or disorder.

“Chronic disease” as used refers to a long-lasting condition that can be controlled but not cured.

As various changes could be made in the above-described materials and methods without departing from the scope of the invention, it is intended that all matter contained in the above description and in the examples given below, shall be interpreted as illustrative and not in a limiting sense.

### EXAMPLES

The following examples are included to demonstrate various embodiments of the present disclosure. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1: A Synthetic Chronogenetic Therapy Gene Circuit for Temporal Drug Delivery

Chronotherapy, the delivery of therapeutic interventions personalized to patient's circadian rhythms, has shown enhanced therapeutic efficacy and reduced side-effects. Currently, many drugs taken in the United States target the product of a circadian gene and therefore there is a need to focus on the timing of drug delivery. In rheumatoid arthritis, patients exhibit diurnal changes in cytokines that lead to inflammatory flares and enhanced disease severity in the early morning. There has been important work showing the administration of anti-inflammatory treatments in the early morning, immediately before the inflammatory flare, in reducing symptoms of RA. Using synthetic biology, we developed chronotherapy-based gene circuits, chronogenetic therapies, that produce our prescribed transgene downstream of the core circadian clock component, Per2. These lentiviral chronogenetic therapies were transduced into murine induced pluripotent stem cells and developed tissue-engineered cartilage as our model system for timed drug delivery. Our anti-inflammatory chronogenetic circuit was capable of producing interleukin-1 receptor antagonist (IL-1Ra) in an oscillatory manner tracking with circadian rhythms in vitro. Additionally, the tissue engineered pellets were able to entrain to host circadian rhythms when implanted into mice and produce differing levels of IL-1Ra in the serum at different times of day. The chronogenetic synthetic gene circuits provide a novel cell therapy driving by the circadian clock for controlled biologic delivery at prescribed times of day.

Chronotherapy, or the delivery of therapeutic interventions based on the patient's circadian rhythm, is emerging as an important method for minimizing side effects and increasing efficacy of drugs. Circadian rhythms are driven by the circadian clock, an internal genetic timing mechanism that operates on a roughly 24-hour period and exists within the brain and nearly all cells in peripheral tissues. The core clock genes, comprised of transcriptional activators Bmal1 and Clock and transcriptional repressors Per1/2 and Cry1/2 and output genes, such as Rev-erbs, Dbp, and Nfil 3, create an auto-regulatory negative feedback loop that drives the oscillatory and timed behavior of the clock. These core clock genes drive expression of many other tissue-specific clock-controlled genes that help maintain tissue homeostasis. Recently, it was discovered that around 50% of mammalian genes are expressed with 24-hour rhythms and their expression is regulated in some way by the circadian clock. Therefore, there has been an increase in research trying to understand the importance of therapeutic delivery and matching it to circadian rhythms.

Recently it was found that over half (56) of the top 100 selling drugs in the United States target the product of a circadian gene (64). To this end, there are research and clinical trials looking into timed delivery of drugs for increasing their efficacy and minimizing side effects. Studies have shown that time of day is critical to outcomes in heart surgery, cardiovascular disease treatment, cancer treatment, as well as others.

In musculoskeletal diseases, such as rheumatoid arthritis (RA) and osteoarthritis (OA), there are diurnal expression patterns in cytokines that demonstrate circadian patterns in the intensity of symptoms, with inflammatory flares occurring in the early morning. There is great evidence for the importance of chronotherapy in patients with RA. In trials of non-steroidal anti-inflammatory drugs (NSAIDs) or methotrexate, patients that took these drugs at night showed improved outcomes and reduced adverse effects to the drugs. Additionally, patients who received drugs at night instead of in the morning, to combat the increase in IL-6 in the early morning, reported reduced joint pain and inflammation. Therefore, there is a need for intermittent or timed delivery of biologics at specific times of day for enhanced efficacy in musculoskeletal diseases such as RA.

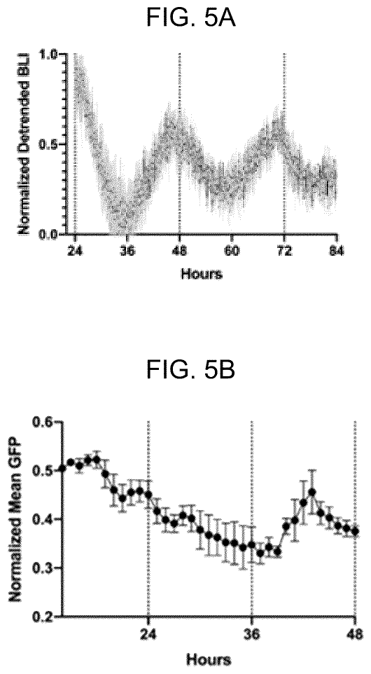

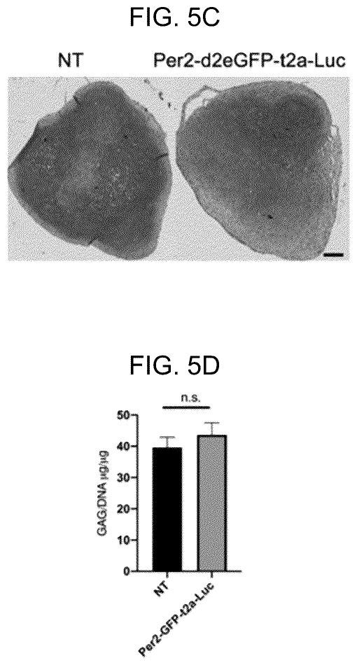

Unfortunately, this process of taking drugs at specific times of day can be difficult to maintain since it requires constant daily injections or delivery of drugs at inconvenient times, such as the case of arthritis where delivery of the drug in the early morning immediately before an inflammatory flare would be best. However, with recent advances in synthetic biology and genetic engineering, cell-based therapies that harness cells for localized, controlled, and long-lasting delivery of prescribed biologic drugs, can be a promising approach to ensure drug delivery at specific times of day and over specific frequencies. To this end, the goal of this study was to create cell-based chronotherapy gene circuits, chronogenetic therapies, that use the cell's own biologic circadian rhythm to provide gene-based delivery of biologic drugs at prescribed times and frequencies. Therefore, we developed two lentiviral synthetic gene circuits driven by the core clock gene, Per2, to produce a destabilized green fluorescence protein (d2eGFP) as an initial proof-of-concept reporter circuit (Per2-d2eGFP-t2a-Luc) and interleukin-1 receptor antagonist (IL-1Ra) as an anti-inflammatory approach to target RA (Per2-IL1Ra-t2a-Luc). All circuits contained a 2A linker to produce luciferase in addition to the therapeutic drug for circadian monitoring. We first examined the ability of the GFP reporter circuit to produce protein in an oscillating manner in transduced murine induced pluripotent stem cells (miPSCs) differentiated into tissue-engineered cartilage. Tissue-engineered cartilage was used as the model delivery system due to the fact that its avascular and aneural and can easily be implanted and survive in any location (23, 61). We then worked to characterize the Per2-IL1Ra-t2a-Luc circuit in vitro to ensure oscillating production of IL-1Ra and to determine if this circuit could maintain its circadian clock in the presence of inflammatory cytokines, which has previously been shown to disrupt the clock (33). We then worked to test our Per2-IL1Ra-t2a-Luc circuit in vivo to understand tissue-engineered cartilage circadian clock entrainment to the host and the ability for timed drug delivery and IL-1Ra concentrations in the serum (FIG. 5.1). We show the ability of these chronogenetic circuits to deliver our therapeutic biologics at specific times of day, driven by Per2 expression both in vitro and in vivo. This approach is the very first creation of a cell-based therapy for chronotherapy applications through the use of synthetic biology and tissue engineering of stem cell gene circuits.

### Results

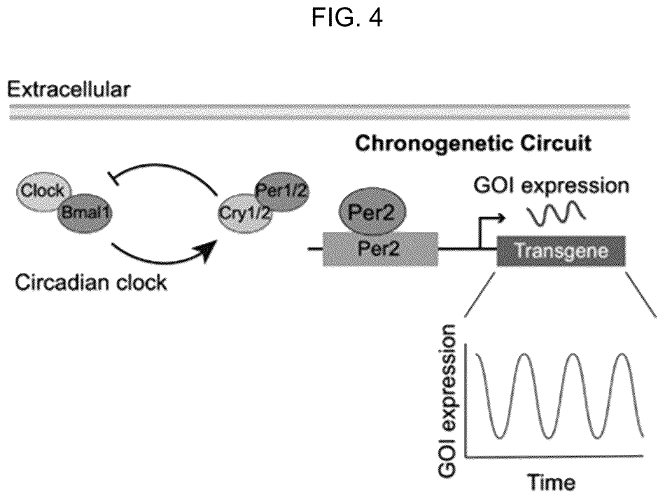

Characterization of chronogenetic reporter circuits and tissue engineered cartilage: To first evaluate the ability to create chronogenetic circuits, the ability of our synthetic circuits to drive production of both bioluminescence and a destabilized green fluorescence protein reporter (d2eGFP) in an oscillating manner was assessed. We found that our Per2-d2eGFP-t2a-Luc circuit had oscillating expression of luminescence (n=3, period=22.73±1.31 h, FIG. 4A) and d2eGFP protein expression (n=5, period=25±1.4 h, FIG. 4B), showing that our chronogenetic circuit concept is capable of driving timed expression of a desired output. Additionally, tissue engineered cartilage was evaluated for sulfated glycosaminoglycans (sGAG), a main component of the cartilage extracellular matrix. Non-transduced (NT) pellets and Per2-d2eGFP-t2a-Luc pellets were stained with safranin-O and both showed rich red stain for sGAGs (n=4/group, FIG. 4C). Additionally, sGAG content was quantified and normalized to DNA content within each pellet (n=7/group, FIG. 4D). There were no significant differences in sGAG content between groups, showing that our lentiviral chronogenetic therapy did not impact tissue-engineered cartilage formation. Therefore, our reporter chronogenetic circuit, Per2-d2eGFP-t2a-Luc, showed protein production driven by the circadian clock at prescribed times of day.

Synthetic chronogenetic IL-1Ra circuit produces IL-1Ra in a circadian manner: After seeing that our reporter circuit functioned well, we sought to test the ability of our Per2-IL1Ra-t2a-Luc chronogenetic circuit to produce IL-1Ra at different concentrations over time, with the overall goal of creating a circuit capable of inhibiting inflammatory flares at specific times of day. Per2-IL1Ra-t2a-Luc pellets showed circadian oscillations driven by Per2 (n=3, period=22.19±1.27 h, FIG. 5A). Additionally, it has been reported that inflammatory cytokines such as IL-1 can disrupt the circadian clock in cartilage. Therefore, we tested the ability of our Per2-IL1Ra-t2a-Luc circuit to maintain its oscillations in the presence of IL-1 and saw the maintenance of circadian oscillations after the addition of IL-1 (n=2, period=20.89±1.04 h, FIG. 5B). The maintenance of oscillations even in the presence inflammation provides additional support for the ability to use this circuit as a cell-based therapeutic to target inflammatory flares.

To determine the production of IL-1Ra, our therapeutic drug, we collected the media from Per2-IL1Ra-t2a-Luc pellets and collected the pellets for gene expression every 3 hours for 72 hours. Per2-IL1Ra-t2a-Luc pellets had increased Illrn gene expression compared to NT controls (n=6/group, p<0.001, FIG. 5C). Additionally, Illrn expression in our chronogenetic circuit showed circadian oscillations and a significant difference between one peak and trough of the Illrn expression (period=22.8±3.6 h, p<0.05, FIG. 5C). Importantly, IL-1Ra production followed a similar oscillating trend to Illrn gene expression and maintained a roughly 24-hour period of peak IL-1Ra expression driven by the circadian clock (n=6, period=22.5±2.9 h, FIG. 5D). Additionally, there were significant peaks of IL-1Ra production (33, 63, and 66 h, p<0.04) compared to troughs in IL-1Ra expression (42, 54, 57, 72 h, p<0.01, FIG. 5D). Excitingly, this showed that our chronogenetic anti-inflammatory therapy had the ability to produce IL-1Ra at different times of day in vitro.

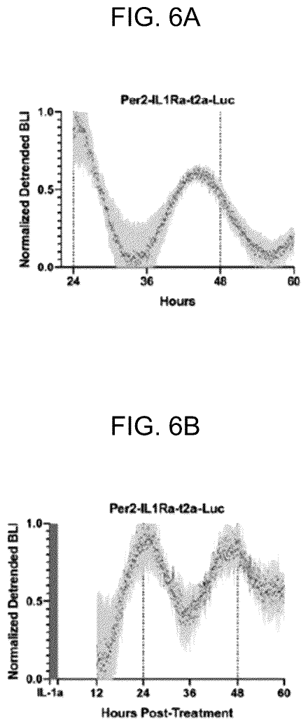

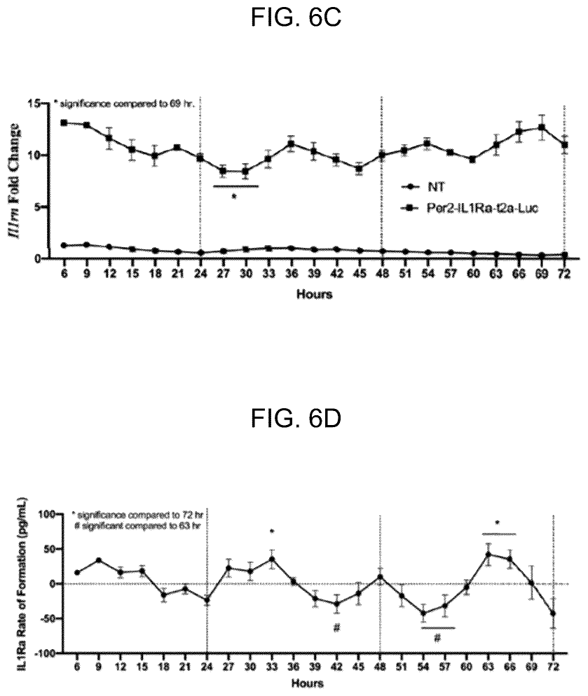

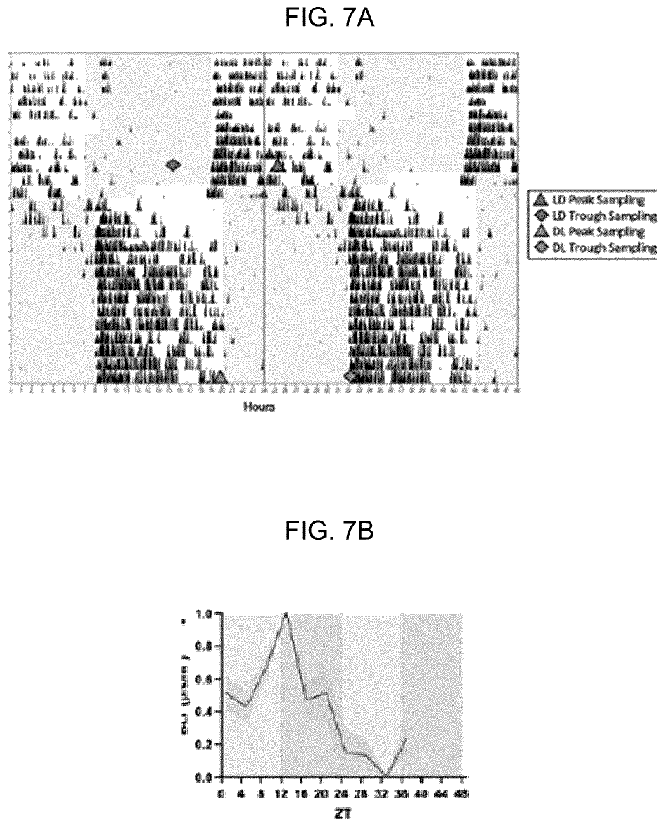

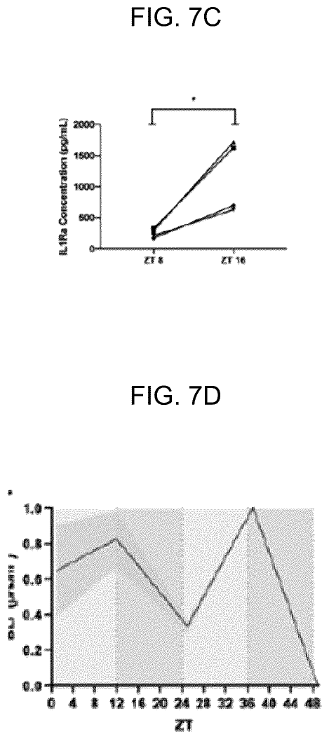

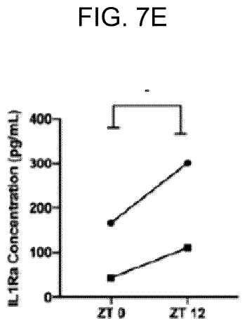

Chronogenetic IL-IRa therapy circuits are capable of delivering IL-Ira at different times of day and can entrain to the circadian clock of the host in vivo: However, we wanted to test the effectiveness of this chronogenetic therapy approach in vivo both in our delivery mechanism and its ability to synchronize to the circadian clock of the host and in its ability to deliver therapeutic proteins at prescribed times. Therefore, we implanted the Per2-IL1Ra-t2a-Luc pellets into C57 mice in the flank. The bioluminescence of the pellets was monitored via around the clock (36 h) bioluminescence intensity (BLI) imaging three days following implant and was found to be oscillatory, peaking at ZT13 (n=4, FIG. 6B). Following the confirmation of circadian Per2-Luc gene expression in vivo, we assayed for IL-1Ra levels in serum through serial blood draws at 4 h post BLI peak and trough, to account for the time to translation of IL-1Ra following peak Per2 promoter activation (FIG. 6A). IL-1Ra concentration was significantly increased in the serum at 4 h following the peak time of BLI compared to the trough of Per2 expression (n=4, p=0.0342, FIG. 6C).

To assess entrainment of the implanted pellets to the mouse, the lighting schedule was reversed from standard 7a lights on/7p lights off to 8a lights off/8p lights on. For a week prior to this transition and for a month following, mouse wheel-running actigraphy was tracked to monitor circadian entrainment to the light cycle. Within one-week post-delay, all mice were once again active during the dark phase and inactive in the light (FIG. 6A). Following confirmation of re-entrainment via wheel running, the BLI of the implanted pellets was reassessed. Bioluminescence imaging of pellets showed pellet entrainment to mouse circadian activity, with BLI peaking at ZT13, as observed previously (FIG. 6D). Serum was again collected for IL-1Ra concentration 12 hours apart and IL-1Ra concentration was significantly increased during peak expression of Per2 than during trough expression (n=2, p=0.046, FIG. 6E). Taken together, these results show the ability of our synthetic circuit to deliver IL-1Ra at different times of day, tracking with the clock component Per2, and the ability of our drug delivery system, a tissue engineered pellet, to entrain to the host for prescribed time of day treatment.

### Discussion

Going off the principles of chronotherapy, we used synthetic biology to develop cell-based chronogenetic therapies that are capable of delivering biologic drugs at prescribed times, tracking with circadian rhythms. To our knowledge, this is the first approach to create a cell therapy for timed drug delivery. While the notion of taking drugs in line with circadian rhythms is gaining popularity, the inconvenient times and need to take the drugs at the same time each day makes it difficult for patients to adhere to the chronotherapy approach. By harnessing the power of stem cells and creating a cell-therapy, we created a mechanism to deliver drugs at specific times without any burden on the patient that can also provide a more localized and controlled delivery of the drug. This approach can be used for disease targeting where symptoms or disease severity changes over the day and can be used for more effective delivery of growth factors or drugs targeting tissue homeostasis.

By developing an in vitro system to characterize our chronogenetic circuits we can create model systems to better understand the effect of chronotherapy on drug delivery and disease targets. Although circadian rhythms, clock-controlled genes, and their therapeutic counterparts are starting to be better understood, there are still many unknowns on the effect the core circadian clock has on specific genes, and the importance of timed drug delivery. Therefore, these synthetic circuits and in vitro characterization techniques can be used to better understand important connections between circadian rhythms and disease progression. Additionally, these systems can be used to deliver anabolic factors to cells to create better tissue engineered systems, as well as can be used to create in vitro model systems that can accurately depict cyclic disease states to better understand the effect of time.

Here we show that our anti-inflammatory chronogenetic circuit was capable of delivering the anti-inflammatory cytokine IL-1Ra in a timed and oscillating manner through different concentrations of protein in the serum. This system can be expanded beyond applications in RA and can be used for any inflammatory disease where there are inflammatory flares driven by the circadian clock. Additionally, this type of system can be used to deliver different concentrations of drugs at different times. Importantly, we also evaluated the ability of our tissue engineered cell therapy to entrain to its host, as this is important when producing chronogenetic therapies driven by the host circadian clock. Here we see that our tissue engineered constructs synchronize to the host and can continue to entrain even with shifts in circadian rhythms. The ability of the tissue engineered system to shift along with the mouse circadian clock is important in the ability of our tissue engineered systems to maintain prescribed drug delivery synchronized to personalized circadian rhythm even during perturbations or shifts in circadian rhythms that can happen with shift work, circadian disorders, and other disease pathologies that can affect the circadian clock.

Interestingly, we saw that our anti-inflammatory chronogenetic circuit was capable of maintaining its circadian rhythm in the presence of inflammatory cytokine, IL-1. Previously, it has been reported that inflammatory cytokines disrupt the circadian clock but that the administration of anti-inflammatory agents could restore the circadian clock. Therefore, our chronogenetic circuit is capable of being clock-preserving, which is important for its ability to deliver anti-inflammatory biologics at prescribed times of day. Additionally, this type of clock-preservation can help elucidate the relationship between circadian rhythms and inflammation.

The creation of these chronogenetic therapies opens up a whole new field of cell-based therapies and ways to enhance drug efficacy and mitigate side effects through the timed delivery of drugs personalized to the patient's circadian rhythm. The chronogenetic circuits developed here were all driven by core clock gene Pert, however other core clock genes and the thousands of tissue-specific clock-controlled genes can be used for alternative timed delivery or for specific inputs. Additionally, multiple circuits can be used with antiphase drivers to create a two-pronged approach with one drug delivered at multiple peaks throughout the day or for two different drugs to be delivered at different times, depending on the need in disease progression. Additionally, mutations can be made in core clock genes to delay or advance the phase of the circadian clock, to tailor this approach to any timing of delivery.

Using this type of circadian clock driven chronogenetic therapy approach we can deliver drugs at specific times of day to enhance drug efficacy. This work can be directly used to targeting the inflammatory flares associated with RA. However, these approaches can also be expanded to create chronogenetic therapies for many different tissues and targeting different diseases where there are therapeutic targets for clock-controlled genes. Together, this framework for developing personalized chronogenetic therapies is a novel approach to establish a new generation of cell therapies that provide precision and effective therapeutic drug delivery.

Although the main focus of these circuits was for use in tissue-engineered cartilage and targeting arthritis, these principles can be used to create smart cell therapies in any tissue or any field. Within cartilage, the therapeutic transgene can be replaced with a biologic to modulate arthritis pain or promote anabolic pathways to promote tissue regeneration. Additionally, these types of systems can be expanded to other musculoskeletal tissues. For example, we've shown a system in muscle cells to be able to sense and inhibit muscle fibrosis. These systems can be further expanded beyond musculoskeletal tissues and can be used in any diseases involving inflammation, mechanical stimuli, maintenance of the circadian clock, and/or requiring timed drug delivery. These systems will also further our understanding of important disease processes and can elucidate unknowns in disease pathologies. Finally, this work adds to the synthetic biology molecular toolkit of approaches to create better and more effective therapies to treat disease.

### Methods

Murine induced pluripotent stem cell culture and differentiation: Murine induced pluripotent stem cells (miPSCs) were derived from tail fibroblasts from adult C57BL/6 mice and validated for pluripotency as described previously. miPSCs were cultured in Dulbecco's Modified Eagle's Medium High Glucose (DMEM-HG, Gibco), 20% lot selected fetal bovine serum (FBS, Atlanta Biologicals), 100 nM minimum essential medium nonessential amino acids (NEAA, Gibco), 55 μM 2-mercaptoethanol (2-me, Gibco), 24 ng/ml gentamicin (Gibco), and 1000 U/mL mouse leukemia inhibitory factor (LIF, Millipore), and maintained on mitomycin C-treated mouse embryonic fibroblasts (Millipore).

miPSCs were differentiated toward a mesenchymal state using a high-density micromass culture in differentiation medium containing DMEM-HG, 1% culture medium supplement containing recombinant human insulin, human transferrin, and sodium selenite (ITS+, Corning), 100 nM NEAA, 55 UM 2-me, 24 ng/ml gentamicin, 50 μg/mL L-ascorbic acid, and 40 μg/mL L-proline. On days 3-5, this medium was supplemented with 100 nM dexamethasone and 50 ng/ml bone morphogenetic protein 4 (BMP-4; R&D Systems). After 15 days, the micromasses were dissociated with pronase (Millipore Sigma) and collagenase type II (Worthington Biochemical) and the pre-differentiated iPSCs (PDiPSCs) were plated on gelatin-coated dishes in expansion medium containing DMEM-HG, 10% lot-selected FBS, 1% ITS+, 100 nM NEAA, 55 UM 2-me, 1% penicillin/streptomycin (P/S, Gibco), 50 μg/mL L-ascorbic acid, 40 μg/mL L-proline, and 4 ng/ml of basic fibroblast growth factor (bFGF, R&D Systems).

To create tissue engineered cartilage, passage 2 PDiPSCs were pelleted by centrifugation of 250K cells (49). Pellets were cultured for 14-21 days in chondrogenic medium consisting of DMEM-HG, 1% ITS+, 100 nM NEAA, 55 μM 2-me, 1% P/S, 50 μg/mL L-ascorbic acid, 40 μg/mL L-proline, 100 nM dexamethasone, and 10 ng/ml TGF-I33 (R&D Systems).

Cell-based chronotherapy circuit design: We developed two lentiviral systems activated by Per2 expression, a core circadian clock gene. All circuits contained a t2a component that linked luciferase (Luc) downstream of the transgene, with the goal of creating a chronotherapy circuit that had its own luciferase reporter system. Therefore, upon activation of the repressive arm of the circadian clock and subsequent activation of Per2, the chronotherapy circuits would be activated and produce either a short-lived green fluorescence protein (Per2-d2eGFP-t2a-Luc) or interleukin-1 receptor antagonist (Per2-IL1Ra-t2a-Luc) over a 24-hour period.

Per2-d2eGFP-t2a-Luc circuit—The murine Per2 promoter was obtained from a lentiviral Per2-Luc plasmid. This promoter was cloned in place of the CMV promoter in a CMV-GFP-t2a-Luc lentiviral cassette purchased from Systems Biosciences. The CMV promoter was removed with restriction enzymes and the Per2 promoter was incorporated using Gibson Assembly. A destabilized green fluorescence protein (d2eGFP) was obtained [No. 26821, Addgene] and cloned in place of the stable GFP in the backbone cassette using restriction enzymes and Gibson Assembly to create the Per2-d2eGFP-t2a-Luc circuit. The d2eGFP transgene came from the pcDNA3.3_d2eGFP plasmid which was a gift from Derrick Rossi. Therefore, a dual reporter chronotherapy gene circuit was created that would express both short-lived GFP and luciferase downstream of Per2 activation for initial concept characterization.

Per2-IL1Ra-t2a-Luc circuit-Murine interleukin-1 receptor antagonist (IL-1Ra) was incorporated into the Per2-d2eGFP-t2a-Luc cassette in place of the d2eGFP through restriction enzyme removal of d2eGFP and Gibson Assembly to incorporate IL-1Ra. The IL-1Ra was obtained from a plasmid created to sense and respond to inflammation. This lentiviral circuit was then able to produce IL-1Ra and luciferase in response to Per2 activation (Per2-IL1Ra-t2a-Luc) and can be used as an anti-inflammatory therapeutic.

Lentivirus production and cell transduction: Human embryonic kidney (HEK) 293T cells were co-transfected with second-generation packaging plasmid psPAX2 (No. 12260; Addgene), the envelope plasmid pMD2.G (No. 12259; Addgene), and the expression transfer vector (Per2-d2eGFP-t2a-Luc or Per2-IL1Ra-t2a-Luc) by calcium phosphate precipitation to make vesicular stomatitis virus glycoprotein pseudotyped lentivirus. The lentivirus was harvested at 24- and 48-hours post transfection and stored at −80° C. until use. The functional titer of the virus was determined with quantitative real-time polymerase chain to determine the number of lentiviral DNA copies integrated into the genome of transduced HeLa cells. PDiPSCs were transduced before being pelleted. For PDiPSC transductions, virus was thawed on ice and diluted in medium to obtain the desired number of viral particles to achieve a multiplicity of infection of 3. Polybrene was added to a concentration of 4 μg/mL to aid in transduction. The medium of the cells was aspirated and replaced with virus-containing medium, and cells were incubated for 24 hours before aspirating the viral medium.

In vitro characterization of chronogenetic therapy circuits: All chronogenetic therapy circuits were characterized in vitro once the cells had become tissue engineered cartilage pellets after 14 days in chondrogenic culture. Both circuits were subject to bioluminescence recordings to track circadian oscillations. Per2-d2eGFP-t2a-Luc constructs were also subject to fluorescence imaging to quantify GFP expression over time. Media from the Per2-IL1Ra-t2a-Luc pellets was collected every 3 hours for 72 hours to determine IL-1Ra production, and pellets were harvested every 3 hours for 72 hours for IL1rn gene expression.

Bioluminescence recordings: Tissue engineered cartilage pellets (Per2-d2eGFP-t2a-Luc or Per2-IL1Ra-t2a-Luc) were plated in 35 mm petri dishes with 1 mL of recording medium containing D-luciferin (Goldbio), sealed with vacuum grease, and placed in a light-tight 36° incubator containing photo-multiplier tubes (PMTs) (Hamamatsu Photonics). Each dish was placed under one PMT and the bioluminescence was recorded as photons per 180 seconds. Bioluminescence data were detrended with a 24 h moving average and analyzed in ChronoStar 1.0 (182). Recoding medium contained DMEM powder (Sigma), B27 supplement (Invitrogen), P/S, L-glutamine (Invitrogen), HEPES (Sigma), and D-glucose (Invitrogen).

Fluorescence imaging: After 14 days of chondrogenic culture Per2-d2eGFP-t2a-Luc pellets were digested with collagenase type II (Worthington Biochemicals), cast in a thin layer of agarose at a density of 10 million cells/mL on glass bottom black 96-well plates (Grier Bio), and cultured in chondrogenic medium. Plates were placed in a Cytation5 plate reader (BioTek) and agarose was subjected to fluorescence imaging at 4× magnification for 48 hours with images taken every hour. Image sequences were concatenated into videos using Gen5 software and individual cells were visualized and traced for GFP output.

Enzyme-linked immunosorbent assays: Media from Per2-IL1Ra-t2a-Luc pellets was collected every 3 hours for 72 hours and stored at −20° C. IL-1Ra concentration was measured with DuoSet enzyme-linked immunosorbent assay (ELISA) specific to mouse IL-1Ra/IL-1F3 (R&D Systems). Data are reported as rates of formation with a 6 h moving average.

Histological and biochemical analysis of tissue engineered cartilage: After 14 days of chondrogenic culture pellets were washed with PBS and fixed in 10% NBF for 24 hours, paraffin embedded, and sectioned at 8 μm thickness. Slides were stained for Safranin-O/hematoxylin/fast green.

For biochemical analysis, pellets were digested overnight in 125 μg/mL papain at 65° C. DNA content was measured with PicoGreen assay (Thermo Fisher) and total sulfated glycosaminoglycan (sGAG) content was measured using a 1,9-dimethylmethylene blue assay at 525 nm wavelength.

Inflammatory challenge: Per2-IL1Ra-t2a-Luc pellets underwent inflammatory challenge with 1 ng/ml IL-1a. Pellets were placed in recording medium and bioluminescence was recorded for 72 hours. After 72 hours of recording, cytokine was added to the dish and bioluminescence was recorded for an additional 72 hours.

In vivo characterization of chronogenetic therapy circuits: Per2-IL1Ra-t2a-Luc pellets were implanted into mice subcutaneously for circuit characterization and evaluation of therapeutic effect. 4 pellets per group were implanted into adult C57B/6 mice and were subject to evaluation. All procedures were approved by IACUC at Washington University in St. Louis.

Per2-IL1Ra-t2a-Luc pellets were characterized based on ability to entrain to mouse circadian rhythms through mouse actigraphy collection and bioluminescence imaging. Additionally, the ability for the Per2-IL1Ra-t2a-Luc circuit was evaluated through IL-1Ra serum concentration.

Mice were subjected to normal light/dark cycle and wheel running activity was collected. Bioluminescence was imaged every 4 hours for 36 hours. After bioluminescence imaging, pellet peak and trough were determined to be ZT 13 and ZT 5 respectively. To account for IL-1Ra translation, blood draws were taken from mice 4 hours after luminescence peak through cheek stab and serum collection. IL-1Ra serum levels were assessed by ELISA (Quantikine-IL1Ra, R&D Systems).

To evaluate if pellets have the ability to continue to entrain to mice circadian rhythm, mice were phase delayed 12 h using a reversed light/dark schedule. Actigraphy data was collected continuously to monitor entrainment of activity and after 14 days, pellets were imaged for bioluminescence every 12 hours for 48 hours at ZT1 and ZT13. Additionally, serum was collected at the new peak and trough of bioluminescence expression and IL-1Ra serum was quantified by ELISA.

1. A recombinant nucleic acid molecule comprising at least one transcriptional regulatory nucleic acid sequence of a circadian-responsive gene operably linked to a nucleic acid sequence encoding a therapeutic biologic.

2. The nucleic acid molecule of claim 1, wherein the at least one transcriptional regulatory nucleic acid sequence is a promoter, enhancer, and/or repressor nucleic acid molecule corresponding to a promoter, repressor, and/or enhancer region of a circadian-responsive gene.

3. The nucleic acid molecule of claim 2, wherein the at least one transcriptional regulatory sequence of a circadian-responsive gene is selected from one or more of a Bma/1 gene promoter, a GSK3β gene promoter, NPAS2 gene promoter, Clocks gene promoter, Cry1 gene promoter, a Cry2 gene promoter region, a Per1 gene promoter region, a Per2 gene promoter region, a Per3 gene promoter region, Dec1 gene promoter region, a Dec2 gene promoter region, a RORa gene promoter region, ROR/3 gene promoter region, a REV-ERBa gene promoter region, Dbp gene promoter region, a CK fa gene promoter region, a CK18 gene promoter region, Nfi/3 gene promoter region and any combination thereof.

4. The nucleic acid molecule of claim 2, wherein the at least one transcriptional regulatory nucleic acid sequence comprises one or more D box regions, E box regions, RRE regions and any combination thereof.

5. The nucleic acid molecule of claim 1, wherein transcriptional regulatory region comprises one or more E-box domains which the transcription factor BMAL1 and/or CLOCK bind.

6. The nucleic acid molecule of claim 1, wherein the transcriptional regulatory region comprises one or more PER and/or CRY repressor elements.

7. The nucleic acid molecule of claim 1, wherein the transcriptional regulatory region comprises one or more ROR response elements.

8. The nucleic acid molecule of claim 1, wherein the transcriptional regulatory region comprises one or more RRE elements.

9. The nucleic acid molecule of claim 1, wherein the transcriptional regulatory region comprises one or more D-box elements upon which DBP and NFIL3 dimers bind.

10.-23. (canceled)

24. The nucleic acid molecule of claim 1, wherein the therapeutic biologic nucleic acid sequence encodes an anti-catabolic polypeptide, an anti-inflammatory polypeptide, a pro-anabolic polypeptide, a pro-regenerative polypeptide, an anti-microbial polypeptide, an anti-pain polypeptide, a morphogen, a growth factor, an anti-cancer nucleic acid or an anti-cancer polypeptides.

25. The nucleic acid molecule of claim 24, wherein the therapeutic biologic nucleic acid molecule encodes for one or more of IL-1 Ra, sTNFR1/2, IL-10 IL-4, a growth factor from the TGFβ superfamily, IGF, CTGF, FGF, PDGF, a kappa opioid ligand pro-peptide (e.g., prodynorphin), a mu/delta opioid ligand pro-peptide (e.g. proenkephalin), a delta/mu opioid ligand pro-peptide (proopiomelanocortin), an endocannabinoid ligand synthesis driver, TNF, IL-7, IL-15, IL-12, IL-2, IFN, NOS, PTGIS, Decorin, TGFβ-receptor, MMP, ALDH2, NR3C1 and any combination thereof.

26. A nucleic acid construct or vector comprising the nucleic acid molecule of claim 1.

27. The vector of claim 26, wherein the vector is a viral vector or the construct is a plasmid.

28. The viral vector of claim 27, wherein the viral vector is an adeno-associated viral vector, a lentiviral vector, or a retroviral vector.

29. A viral particle comprising the viral vector of claim 28.

30. The nucleic acid construct or vector of claim 26, wherein the promoter is a Per2 promoter and the therapeutic biologic is an IL-1 receptor antagonist.

31. A genetically modified cell comprising a heterologous nucleic acid sequence incorporated into its genome, wherein the heterologous nucleic acid sequence comprises the nucleic acid molecule of claim 1.

32.-62. (canceled)

63. A method of treating a condition, disease or disorder in a subject in need thereof, the method comprising administering an effective amount of a composition comprising the genetically modified cell of claim 31.

64. The method of claim 63, wherein the condition, disease or disorder is a chronic inflammatory disease, an acute inflammatory disease, a degenerative disease of any tissue or organ, chronic or acute pain, cancer, cardiovascular disease, osteoarthritis, cardiac hypertrophy, atherosclerosis, asthma, irritable bowel syndrome, muscle atrophy, angina, atrial fibrillation, hypertension, intimal hyperplasia, valve disease, scleroderma, achalasia, volvulus, diabetic nephropathy, glomerulosclerosis, cerebral edema, hydrocephalus, migraine, stroke, glaucoma, ankylosing spondylitis, carpal tunnel syndrome, chronic back pain, dupytre's contracture, osteoporosis, rheumatoid arthritis, collagenopathies, Musculo dystrophies, osteochondroplasias, polycystic kidney disease, ARDS, emphysema, pulmonary fibrosis, ventilator injury, pre-eclampsia, sexual dysfunction, and urinary incontinence.
